SEQUENCE LISTING (19 sequences)

SEQ ID NO: 1 - protein, 4 aa, Artificial Sequence
  source: /note=“Description of Artificial Sequence Synthetic peptide”
DAHK

SEQ ID NO: 2 - protein, 4 aa, Artificial Sequence
  source: /note=“Description of Artificial Sequence Synthetic peptide”
DTHK

SEQ ID NO: 3 - protein, 4 aa, Artificial Sequence
  source: /note=“Description of Artificial Sequence Synthetic peptide”
AKDH

SEQ ID NO: 4 - protein, 14 aa, Artificial Sequence
  source: /note=“Description of Artificial Sequence Synthetic peptide”
GVFRRDAHKSEVAH

SEQ ID NO: 5 - protein, 23 aa, Artificial Sequence
  source: /note=“Description of Artificial Sequence Synthetic peptide”
SAYSRGVFRRDAHKSEVAHRFKD

SEQ ID NO: 6 - protein, 14 aa, Artificial Sequence
  source: /note=“Description of Artificial Sequence Synthetic peptide”
VFRREAHKSEIAHR

SEQ ID NO: 7 - protein, 4 aa, Artificial Sequence
  source: /note=“Description of Artificial Sequence Synthetic peptide”
EAHK

SEQ ID NO: 8 - protein, 4 aa, Artificial Sequence
  source: /note=“Description of Artificial Sequence Synthetic peptide”
DAHR

SEQ ID NO: 9 - protein, 4 aa, Artificial Sequence
  source: /note=“Description of Artificial Sequence Synthetic peptide”
DARK

SEQ ID NO: 10 - protein, 5 aa, Artificial Sequence
  source: /note=“Description of Artificial Sequence Synthetic peptide”
RDAHK

SEQ ID NO: 11 - protein, 6 aa, Artificial Sequence
  source: /note=“Description of Artificial Sequence Synthetic peptide”
RDAHKS

SEQ ID NO: 12 - protein, 6 aa, Artificial Sequence
  source: /note=“Description of Artificial Sequence Synthetic peptide”
RRDAHK

SEQ ID NO: 13 - protein, 7 aa, Artificial Sequence
  source: /note=“Description of Artificial Sequence Synthetic peptide”
RRDAHKS

SEQ ID NO: 14 - protein, 7 aa, Artificial Sequence
  source: /note=“Description of Artificial Sequence Synthetic peptide”
RDAHKSE

SEQ ID NO: 15 - protein, 8 aa, Artificial Sequence
  source: /note=“Description of Artificial Sequence Synthetic peptide”
RRDAHKSE

SEQ ID NO: 16 - protein, 10 aa, Artificial Sequence
  source: /note=“Description of Artificial Sequence Synthetic peptide”
FRRDAHKSEV

SEQ ID NO: 17 - protein, 11 aa, Artificial Sequence
  source: /note=“Description of Artificial Sequence Synthetic peptide”
FRRDAHKSEVA

SEQ ID NO: 18 - protein, 609 aa, Homo sapiens [sequence omitted]

SEQ ID NO: 19 - protein, 4 aa, Artificial Sequence
  source: /note=“Description of Artificial Sequence Synthetic peptide”
DAKH

(19) United States
(12) Patent Application Publication
(10) Pub. No.: US 2024/0043794 A1
(43) Pub. Date: Feb. 8, 2024

(54) Chemically Defined Serum Albumin Substitutes

(71) Applicant: Life Technologies Corporation, Carlsbad, CA (US)

(72) Inventors: Joel JESSEE, Stuart, FL (US); David KUNINGER, Frederick, MD (US); Soojung SHIN, Clarksburg, MD (US)

(21) Appl. No.: 18/257,534

(22) PCT Filed: Dec. 15, 2021
(86) PCT No.: PCT/US2021/063618

§ 371 Date: Jun. 14, 2023

Related U.S. Application Data
(60) Provisional application No. 63/125,619, filed on Dec. 15, 2020.

(51) Int. Cl. C12N 5/00; C12N 9/02

(52) U.S. Cl. CPC C12N 5/0037; C12N 9/0089; C12Y 115/01001; C12N 2501/71; C12N 2500/90; C12N 2500/38

(57) ABSTRACT

Provided herein, inter alia, are chemically defined components and compositions that substitute or partially substitute for albumin in cell culture media. The components and compositions may support cell cultures, protect cells, or enhance viability of cultured cells. Further provided are chemically defined culture media supplements for use in cell culture media containing albumin. The chemically defined culture media supplements may rescue cells from albumin-induced toxicity.

### RELATED APPLICATIONS

This application is a 35 U.S.C. 371 national phase of PCT International Application No. US2021/063618, filed on Dec. 15, 2021, which claims the benefit of and priority to U.S. Provisional Application No. 63/125,619 filed on Dec. 15, 2020, the entire contents of each of which is incorporated herein by reference.

### SEQUENCE LISTING

The instant application contains a Sequence Listing which has been submitted electronically in ST.26 XML format and is hereby incorporated by reference in its entirety. Said XML document, created on Jun. 14, 2023, is named TP109059USPCT1_SL.XML and is 26,000 bytes in size.

### BACKGROUND

Albumin, a single polypeptide with a molecular mass of approximately 66 kilodaltons, is the most abundant protein in the serum of vertebrates. Albumin serves as a key component of cell culture, and confers beneficial properties to expanding cells. However, albumin also contributes as major source of variability in cell culture performance. For example, the chemical compositions of albumins vary between lots, even from a single manufacturer. Albumin carries a number of substances from the blood, including hormones, vitamins, and enzymes, and can be contaminated with components that are toxic to cells (e.g., transition metals), or which improve cell viability. All of these contaminants can affect the viability of the cell culture, and cause inconsistencies in biological studies. Additionally, it is difficult to control interactions between media components and albumins, and sequestration of media components by albumin may limit their accessibility to the cultured cells.

In view of the foregoing, chemically defined albumin substitutes for use in cell culture media are highly desirable, as are chemically defined culture media supplements for use in cell culture media containing albumin, e.g. to rescue albumin-induced toxicity. Provided herein are solutions to these and other problems in the art.

### SUMMARY

The embodiments disclosed herein generally relate to compositions comprising chemically defined substitutes or partial substitutes for albumin, and methods of use thereof. The compositions may be culture medium or supplements to support cell cultures, for example of cells that are vulnerable to stress. The compositions may protect cells, for example stem cells, neural cells and oligodendrocytes, from damage caused by processes including freeze/thaw cycles, transportation, and laboratory procedures including transfection. The medium or supplements provided herein may be used in cell culture media including albumin to, for example, rescue cells from albumin-induced toxicity. The medium or supplements provided herein may further enhance viability of cultured cells.

In an aspect a cell culture medium is provided, the cell culture medium including a peptide including superoxide dismutase activity and Cu+, Zn+ chelating activity. In embodiments, the cell culture medium further includes one or more of a vitamin E analog, a hydrogen peroxide reducing reagent, and a superoxide scavenger.

In an aspect is provided a cell culture supplement, the cell culture supplement including a peptide including superoxide dismutase activity and Cu+, Zn+ chelating activity. In embodiments, the cell culture supplement further includes one or more of a vitamin E analog, a hydrogen peroxide reducing reagent, and a superoxide scavenger.

In an aspect is provided a method for growing cells in culture, the method including growing the cells in a cell culture medium provided herein including embodiments thereof.

In an aspect a method of growing cells in culture is provided, the method including growing the cells in a cell culture medium supplemented with a cell culture supplement provided herein including embodiments thereof.

In an aspect is provided a method of rescuing cells from albumin-induced toxicity, the method including contacting a cell exhibiting albumin-induced toxicity with a cell culture supplement provided herein including embodiments thereof.

In an aspect a method for expanding cells in culture is provided, the method including contacting the cells with a serum-free, albumin-free cell culture medium in a cell culture medium provided herein including embodiments thereof.

In an aspect a method for recovering cells from oxidative stress is provided, the method including contacting the cells with a cell culture supplement provided herein including embodiments thereof, or growing the cells in a cell culture medium provided herein including embodiments thereof.

In an aspect is provided a cell culture supplement, the cell culture supplement including: (i) a peptide including superoxide dismutase activity and Cu+, Zn+ chelating activity; (ii) vitamin E or an analog thereof; and (iii) a superoxide scavenger.

In an aspect a cell culture kit is provided, the cell culture kit including a serum-free cell culture medium and a cell culture supplement provided herein including embodiments thereof.

### BRIEF DESCRIPTION OF THE DRAWINGS

FIG. 1A shows that performance differences exist among commercially available albumins when present in various cell cultures grown in B27 media.

FIGS. 1B-1C show main effects plots illustrating the interaction between different B27 culture components and BSA, and the effect of the interactions on neuronal cell viability.

FIGS. 1D-1E show main effects plots illustrating the interaction between different B27 culture components and recombinant human serum albumin (rHSA), and the effect of the interactions on neuronal cell viability.

FIGS. 1F-1J show main effects plots illustrating interactions between different B27 culture components at a broad concentration of rHSA, and the effect of the interactions on neuronal cell viability.

FIG. 2A shows component variation observed between different lots and sources of BSA.

FIG. 2B are bar graphs comparing rat neuron survival in the presence of BSA1 and BSA2 (top panel), and the effect of added iron on rat neuron survival (bottom panel).

FIG. 2C are bar graphs illustrating that addition of antioxidant in culture prevents and/or reverses iron induced toxicity.

FIGS. 2D-2E are bar graphs showing the effect of different albumin sources on stem cell proliferation. Results for mouse embryonic stem cells (mESC) are shown in FIG. 2D and results for human neural stem cells (hNSC) are shown in FIG. 2E.

FIG. 2F shows the effect of albumin from different sources on the expression of forkhead box protein G1 (FOXG1) and Paired box protein (PAX-6) in human pluripotent stem cells grown in Essential 6 medium.

FIG. 3 is a bar graph showing variation in total reducing activity of albumin homologs from different sources.

FIG. 4A is a bar graph showing varying superoxide dismutase (SOD) activity in albumin homologs from different sources.

FIG. 4B is a bar graph showing that Mito-Tempo has SOD activity at a range of concentrations.

FIG. 5 is a bar graph illustrating that Mito-Tempo can reduce cellular stress induced by reactive oxygen species (ROS).

FIG. 6 is a bar graph showing that albumin homologs have catalase activity.

FIG. 7 is a bar graph showing that various albumin homologs have thiol based antioxidant activity

FIG. 8 shows that chemically defined components, including glutathione and lipoic acid, can remove H₂O₂ to improve rat neuron viability in culture.

FIG. 9 is a bar graph showing that addition of copper to the culture medium of rat neurons results in neuronal cell death.

FIG. 10 are bar graphs showing that certain concentrations of Peptide C retain the metal binding property of HSA, while Peptide A and Peptide B do not chelate metals at the concentrations tested.

FIG. 11 is a bar graph showing that the tetrapeptide DARK (SEQ ID NO: 1) (1^{st} lot, 2^{nd} lot, BSA peptide) has metal binding activity similar to HSA, while a scrambled sequence (scramble) does not.

FIG. 12 is a bar graph showing that Peptide C rescues and/or prevents copper induced toxicity in cultured rat neuron cells.

FIG. 13 is a bar graph showing that Peptide C (DARK (SEQ ID NO: 1)) rescues mouse embryonic stem cells from copper induced stress in a concentration dependent manner. Figure discloses SEQ ID NO: 1.

FIG. 14 is a bar graph illustrating that the presence of copper decreases cell viability of HEK-293 cells in culture, and Peptide C rescues cells from copper induced stress. Figure discloses SEQ ID NO: 1.

FIG. 15 is a bar graph showing that various lots and sources of BSA and HSA have varying antioxidant levels and activity, as measured by a Ferric reducing antioxidant power (FRAP) assay.

FIG. 16A is a bar graph showing that albumins from various sources include different levels of the anti-oxidant Vitamin E.

FIGS. 16B and 16C are bar graphs illustrating the effect of Vitamin E (FIG. 16B) and Trolox (FIG. 16C) on Rat cortical neuron (RCN) cell survival.

FIGS. 17A and 17B are bar graphs illustrating the effect of Peptide C on FoxG1 expression in human pluripotent stem cells grown in Essential 6 culture medium, as measured by ICC (FIG. 17A) and qPCR (FIG. 17B).

FIGS. 18A-18B are bar graphs illustrating that rHSA is toxic to HEK-293 cells (FIG. 18A) and HeLa cells (FIG. 18B), and that chemically-defined supplements as disclosed herein rescue HEK-293 and HeLa cells from rHSA induced toxicity.

FIGS. 19A-19B are bar graphs showing that neuron viability (FIG. 19A) and neurite length (FIG. 19B) of primary neurons are enhanced in the presence of a chemically defined supplement as disclosed herein.

FIG. 20 is a graph comparing the effects of recombinant HSA and various Peptide C-derived peptides of various lengths on rat neuron cell growth. Figure discloses SEQ ID NOS 1, 10, 11, 14, 12, 13, and 15-17, respectively, in order of appearance.

FIG. 21 is a graph comparing the effects of recombinant HSA and various peptide C derived-peptides on rat neuron cell growth. The Peptide C derived peptides retain charge properties of Peptide C. Figure discloses SEQ ID NOS 1, 7, 8, 19 and 9, respectively, in order of appearance.

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

### Definitions

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

“Recombinant protein” refers to protein that is encoded by a nucleic acid that is introduced into a host cell. The host cell expresses the nucleic acid. The term “expressing a nucleic acid” is synonymous with “expressing a protein from an RNA encoded by a nucleic acid. “Protein” as used herein broadly refers to polymerized amino acids, e.g., peptides, polypeptides, proteins, lipoproteins, glycoproteins, etc.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 50%, 55%, 60%, 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using a sequence comparison algorithms or by manual alignment and visual inspection. Such sequences that are at least about 80% identical are said to be “substantially identical.” In some embodiments, two sequences are 100% identical. In certain embodiments, two sequences are 100% identical over the entire length of one of the sequences (e.g., the shorter of the two sequences where the sequences have different lengths). In various embodiments, identity may refer to the complement of a test sequence. In some embodiments, the identity exists over a region that is at least about 10 to about 100, about 20 to about 75, about 30 to about 50 amino acids or nucleotides in length. In certain embodiments, the identity exists over a region that is at least about 50 amino acids in length, or more preferably over a region that is 100 to 500, 100 to 200, 150 to 200, 175 to 200, 175 to 225, 175 to 250, 200 to 225, 200 to 250 or more amino acids in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue.

For specific proteins described herein (e.g., HSA), the named protein includes any of the protein's naturally occurring forms, or variants or homologs that maintain the protein activity (e.g., within at least 50%, 80%, 90%, 95%, 96%, 97%, 98%, 99% or 100% activity compared to the native protein). In aspects, variants or homologs have at least 50%, 55%, 60%, 65%, 70%, 75%, 80%, 85%, 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In aspects, the protein is the protein as identified by its NCBI sequence reference. In aspects, the protein is the protein as identified by its NCBI sequence reference or functional fragment or homolog thereof.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., at least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In various embodiments, a comparison window is the entire length of one or both of two aligned sequences. In some embodiments, two sequences being compared comprise different lengths, and the comparison window is the entire length of the longer or the shorter of the two sequences. In certain embodiments relating to two sequences of different lengths, the comparison window includes the entire length of the shorter of the two sequences. In some embodiments relating to two sequences of different lengths, the comparison window includes the entire length of the longer of the two sequences.

Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

Preferred examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (NCBI), as is known in the art. An exemplary BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In certain embodiments, the NCBI BLASTN or BLASTP program is used to align sequences. In certain embodiments, the BLASTN or BLASTP program uses the defaults used by NCBI. In certain embodiments, the BLASTN program (for nucleotide sequences) uses as defaults: a word size (W) of 28; an expectation threshold (E) or 10; max matches in a query range set to 0; match/mismatch scores of 1,-2; linear gap costs; the filter for low complexity regions used; and mask for lookup table only used. In certain embodiments, the BLASTP program (for amino acid sequences) uses as defaults a word size (W) of 3; an expectation threshold (E) of 10;max matches in a query range set to 0; the BLOSUM62 matrix (see Henikoff and Henikoff 1992) Proc. Natl. Acad. Sci. USA 89:10915); gap costs of existence: 11 and extension: 1; and conditional compositional score matrix adjustment.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “isolated”, when applied to a nucleic acid or protein, denotes that the nucleic acid or protein is essentially free of other cellular components with which it is associated in the natural state. It can be, for example, in a homogeneous state and may be in either a dry or an aqueous solution. Purity and homogeneity are typically determined using analytical chemistry techniques such as polyacrylamide gel electrophoresis or high performance liquid chromatography. A protein that is the predominant species present in a preparation is substantially purified.

The term “lipid” as used herein refers to a group of naturally occurring molecules that include fats, waxes, sterols, fat-soluble vitamins (such as vitamins A, D, E, and K), monoglycerides, diglycerides, triglycerides, phospholipids, and others. Lipids may be broadly defined as hydrophobic or amphiphilic small molecules; the amphiphilic nature of some lipids allows them to form structures such as vesicles, multilamellar/unilamellar liposomes, or membranes in an aqueous environment. Biological lipids originate from two distinct types of biochemical subunits isoprene and ketoacyl groups. Lipids may be divided into the following categories: fatty acids, glycerolipids, glycerophospholipids, sphingolipids, saccharolipids, and polyketides (derived from condensation of ketoacyl subunits); and sterol lipids and prenol lipids (derived from condensation of isoprene subunits). Fats are a subgroup of lipids called triglycerides. Lipids also encompass molecules such as fatty acids and their derivatives (including tri-, di-, monoglycerides, and phospholipids), as well as other sterol-containing metabolites such as cholesterol.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

The term “therapeutic cell” as used herein, refers to cells that may be administered to patient or subject in need thereof, in order to effectuate a medicinal effect. Administration may include injection, grafting or implantation into said patient or subject. For example, T-cells may be transplanted into a patient in order to modulate immune responses for treating cancer.

The term “differentiation”, as used herein, refers to a stage in development of the life cycle of a cell.

The phrases “cell culture medium,” “tissue culture medium,” “culture medium” (plural “media” in each case) and “medium formulation” refer to a nutritive solution for cultivating cells or tissues. These phrases can be used interchangeably.

By “cell culture” or “culture” is meant the maintenance or expansion of cells in an artificial, in vitro environment.

The terms “cell culture supplement,” “culture supplement,” or “media supplement” refer to components added to cell culture media to enhance cell expansion. These phrases may be used interchangeably. Cell culture supplements may include one or more of amino acids, salts, peptides, sugars, lipids, vitamins, minerals, metals, and the like. In embodiments, the cell culture supplement includes chemically defined components.

As used here, the term “chemically defined” refers to components of known molecular structures and concentrations.

The term “chemically-defined medium” as used herein refers to medium suitable for in vitro culture of cells, particularly eukaryotic cells, in which all of the chemical components and their concentrations are known.

The term “serum-free” as used herein refers to medium which is free or substantially free of serum. “Substantially free of serum” as used herein refers to media which contains less than about 1% serum by weight, contains only trace amounts of serum, or contains undetectable amounts of serum. In embodiments, substantially free of serum refers to media which contains less than 1% serum by weight, less than 0.95% serum by weight, less than 0.9% serum by weight, less than 0.85% serum by weight, less than 0.8% serum by weight, less than 0.75% serum by weight, less than 0.7% serum by weight, less than 0.65% serum by weight, less than 0.6% serum by weight, less than 0.55% serum by weight, less than 0.5% serum by weight, less than 0.45% serum by weight, less than 0.4% serum by weight, less than 0.35% serum by weight, less than 0.3% serum by weight, less than 0.25% serum by weight, less than 0.2% serum by weight, less than 0.15% serum by weight, less than 0.1% serum by weight, less than 0.09% serum by weight, less than 0.08% serum by weight, less than 0.07% serum by weight, less than 0.06% serum by weight, less than 0.05% serum by weight, less than 0.04% serum by weight, less than 0.03% serum by weight, less than 0.02% serum by weight, or less than 0.01% serum by weight.

The phrase “albumin-free” culture medium refers to culture medium that contains no albumin or is substantially free of albumin. Thus, substantially free of albumin means that albumin is present in less than about 1% (w/v), more preferably less than about 0.1% (w/v), and even more preferably less than about 0.01% (w/v) concentration in the culture medium. Thus, in embodiments, albumin-free culture medium refers to medium which contains less than 1% (w/v) albumin, less than 0.95% (w/v) albumin, less than 0.9% (w/v) albumin, less than 0.85% (w/v) albumin, less than 0.8% (w/v) albumin, less than 0.75% (w/v) albumin, less than 0.7% (w/v) albumin, less than 0.65% (w/v) albumin, less than 0.6% (w/v) albumin, less than 0.55% (w/v) albumin, less than 0.5% (w/v) albumin, less than 0.45% (w/v) albumin, less than 0.4% (w/v) albumin, less than 0.35% (w/v) albumin, less than 0.3% (w/v) albumin, less than 0.25% (w/v) albumin, less than 0.2% (w/v) albumin, less than 0.15% (w/v) albumin, less than 0.1% (w/v) albumin, less than 0.09% (w/v) albumin, less than 0.08% (w/v) albumin, less than 0.07% (w/v) albumin, less than 0.06% (w/v) albumin, less than 0.05% (w/v) albumin, less than 0.04% (w/v) albumin, less than 0.03% (w/v) albumin, less than 0.02% (w/v) albumin, or less than 0.01% (w/v) albumin.

The phrase “synthetically made” or “synthesized” as used herein refers to a molecule that is made by chemical (e.g., protein) synthesis in vitro. Synthesis of peptides is well known in the art. For example and without limitation, peptides may be synthesized using liquid-phase peptide synthesis or solid-phase peptide synthesis. Generally, synthetically made molecules will be purified, for example to remove contaminants and improperly formed molecules (e.g., incomplete or incorrect peptide sequences). Methods of purifying proteins are well known in the art, and include, without limitation, size-exclusion chromatography, ion exchange chromatography (IEC), partition chromatography, high-performance liquid chromatography (HPLC), and reverse-phase chromatography (RPC). In embodiments, a synthetically made peptide is at least 70% pure (contains at least 70% of the desired peptide). In embodiments, a synthetically made peptide is at least 80% pure. In embodiments, a synthetically made peptide is at least 90% pure. In embodiments, a synthetically made peptide is at least 95% pure. In embodiments, a synthetically made peptide is at least 96% pure. In embodiments, a synthetically made peptide is at least 97% pure. In embodiments, a synthetically made peptide is at least 98% pure. In embodiments, a synthetically made peptide is at least 99% pure. Percentages may be based on weight (w/w) or volume (w/v).

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

### Compositions

In an aspect, a cell culture medium is provided, including a peptide having superoxide dismutase activity and Cu+, Zn+ chelating activity.

In embodiments, the peptide has a concentration (is present at a final concentration) between about 25 to about 150 μg/ml. In embodiments, the peptide has a concentration between about 25 μg/mL to about 150 μg/mL. In embodiments, the peptide has a concentration between about 50 μg/mL to about 150 μg/mL. In embodiments, the peptide has a concentration between about 75 μg/mL to about 150 μg/mL. In embodiments, the peptide has a concentration between about 100 μg/mL to about 150 μg/mL. In embodiments, the peptide has a concentration between about 125 μg/mL to about 150 μg/mL.

In embodiments, the peptide has a concentration between about 25 μg/mL to about 125 μg/mL. In embodiments, the peptide has a concentration between about 25 μg/mL to about 100 μg/mL. In embodiments, the peptide has a concentration between about 25 μg/mL to about 75 μg/mL. In embodiments, the peptide has a concentration between about 25 μg/mL to about 50 μg/mL. In embodiments, the peptide has a concentration of about 25 μg/mL, about 50 μg/mL, about 75 μg/mL, about 100 μg/mL, about 125 μg/mL, or about 150 μg/mL.

In embodiments, the peptide has a concentration of about 30 μg/ml to about 125 μg/ml. In embodiments, the peptide has a concentration of about 50 μg/ml to about 100 μg/ml.

In an aspect is provided a cell culture supplement including a peptide having superoxide dismutase activity and Cu+, Zn+ chelating activity. In embodiments, the cell culture supplement is provided as a 5× solution that, when added to culture medium, provides a final peptide concentration of between about 25 to about 150 μg/ml. In embodiments, the cell culture supplement is provided as a 10× solution that, when added to culture medium, provides a final peptide concentration of between about 25 μg/ml to about 150 μg/ml. In embodiments, the cell culture supplement is provided as a 50× solution that, when added to culture medium, provides a final peptide concentration of between about 25 μg/ml to about 150 μg/ml. In embodiments, the cell culture supplement is provided as a 100× solution that, when added to culture medium, provides a final peptide concentration of between about 25 μg/ml to about 150 μg/ml.

In embodiments, when added to culture medium, the cell culture supplement provides a final peptide concentration of between about 25 to about 150 μg/ml. In embodiments, when added to culture medium, the cell culture supplement provides a final peptide concentration of between about 30 μg/ml to 125 μg/ml. In embodiments, when added to culture medium, the cell culture supplement provides a final peptide concentration of between about 50 μg/ml to about 100 μg/ml.

In embodiments, the peptide is synthetically made. In embodiments, the synthetic peptide includes at least 4 amino acid residues. In embodiments, the synthetic peptide includes 4 amino acid residues to 100 amino acid residues. In embodiments, the synthetic peptide includes 8 amino acid residues to 100 amino acid residues. In embodiments, the synthetic peptide includes 12 amino acid residues to 100 amino acid residues. In embodiments, the synthetic peptide includes 16 amino acid residues to 100 amino acid residues. In embodiments, the synthetic peptide includes 20 amino acid residues to 100 amino acid residues. In embodiments, the synthetic peptide includes 24 amino acid residues to 100 amino acid residues. In embodiments, the synthetic peptide includes 28 amino acid residues to 100 amino acid residues. In embodiments, the synthetic peptide includes 32 amino acid residues to 100 amino acid residues. In embodiments, the synthetic peptide includes 36 amino acid residues to 100 amino acid residues. In embodiments, the synthetic peptide includes 40 amino acid residues to 100 amino acid residues. In embodiments, the synthetic peptide includes 44 amino acid residues to 100 amino acid residues. In embodiments, the synthetic peptide includes 48 amino acid residues to 100 amino acid residues. In embodiments, the synthetic peptide includes 52 amino acid residues to 100 amino acid residues. In embodiments, the synthetic peptide includes 56 amino acid residues to 100 amino acid residues. In embodiments, the synthetic peptide includes 60 amino acid residues to 100 amino acid residues. In embodiments, the synthetic peptide includes 64 amino acid residues to 100 amino acid residues. In embodiments, the synthetic peptide includes 68 amino acid residues to 100 amino acid residues. In embodiments, the synthetic peptide includes 72 amino acid residues to 100 amino acid residues. In embodiments, the synthetic peptide includes 76 amino acid residues to 100 amino acid residues. In embodiments, the synthetic peptide includes 80 amino acid residues to 100 amino acid residues. In embodiments, the synthetic peptide includes 84 amino acid residues to 100 amino acid residues. In embodiments, the synthetic peptide includes 88 amino acid residues to 100 amino acid residues. In embodiments, the synthetic peptide includes 92 amino acid residues to 100 amino acid residues. In embodiments, the synthetic peptide includes 96 amino acid residues to 100 amino acid residues.

In embodiments, the synthetic peptide includes 4 amino acid residues to 96 amino acid residues. In embodiments, the synthetic peptide includes 4 amino acid residues to 92 amino acid residues. In embodiments, the synthetic peptide includes 4 amino acid residues to 88 amino acid residues. In embodiments, the synthetic peptide includes 4 amino acid residues to 84 amino acid residues. In embodiments, the synthetic peptide includes 4 amino acid residues to 80 amino acid residues. In embodiments, the synthetic peptide includes 4 amino acid residues to 76 amino acid residues. In embodiments, the synthetic peptide includes 4 amino acid residues to 72 amino acid residues. In embodiments, the synthetic peptide includes 4 amino acid residues to 68 amino acid residues. In embodiments, the synthetic peptide includes 4 amino acid residues to 64 amino acid residues. In embodiments, the synthetic peptide includes 4 amino acid residues to 60 amino acid residues. In embodiments, the synthetic peptide includes 4 amino acid residues to 56 amino acid residues. In embodiments, the synthetic peptide includes 4 amino acid residues to 52 amino acid residues. In embodiments, the synthetic peptide includes 4 amino acid residues to 48 amino acid residues. In embodiments, the synthetic peptide includes 4 amino acid residues to 44 amino acid residues. In embodiments, the synthetic peptide includes 4 amino acid residues to 40 amino acid residues. In embodiments, the synthetic peptide includes 4 amino acid residues to 36 amino acid residues. In embodiments, the synthetic peptide includes 4 amino acid residues to 32 amino acid residues. In embodiments, the synthetic peptide includes 4 amino acid residues to 28 amino acid residues. In embodiments, the synthetic peptide includes 4 amino acid residues to 24 amino acid residues. In embodiments, the synthetic peptide includes 4 amino acid residues to 20 amino acid residues. In embodiments, the synthetic peptide includes 4 amino acid residues to 16 amino acid residues. In embodiments, the synthetic peptide includes 4 amino acid residues to 12 amino acid residues. In embodiments, the synthetic peptide includes 4 amino acid residues to 8 amino acid residues. In embodiments, the synthetic peptide includes 4, 5, 6, 7, 8, 9, 10, 11, 12, 13, 14, 15, 16, 17, 18, 19, 20, 24, 28, 32, 36, 40, 44, 48, 52, 56, 60, 64, 68, 72, 76, 80, 84, 88, 92, 96 or 100 amino acid residues. The synthetic peptide length may be any value or subrange within the recited ranges, including endpoints.

In embodiments, the peptide includes at least one negatively-charged residue. In embodiments, the peptide includes at least one positively-charged residue. In embodiments, the peptide includes one positively-charged residue. In embodiments, the peptide includes two positively-charged residues.

In embodiments, the peptide is selected from DAHK (SEQ ID NO:1), DTHK (SEQ ID NO:2) or EAHK (SEQ ID NO:7). In embodiments, the peptide is DAHK (SEQ ID NO:1). In embodiments, the peptide is DTHK (SEQ ID NO:2). In embodiments, the peptide is EAHK (SEQ ID NO:7). In embodiments, the peptide is an albumin-derived peptide. In embodiments, the peptide is one or more of: DAHK (SEQ ID NO:1), DTHK (SEQ ID NO:2), VFRREAHKSEIAHR (SEQ ID NO:6), EAHK (SEQ ID NO:7), DAHR (SEQ ID NO:8), DARK (SEQ ID NO:9), RDAHK (SEQ ID NO:10), RDAHKS (SEQ ID NO:11), RRDAHK (SEQ ID NO:12), RRDAHKS (SEQ ID NO:13), RDAHKSE (SEQ ID NO:14), RRDAHKSE (SEQ ID NO:15), FRRDAHKSEV (SEQ ID NO:16), or FRRDAHKSEVA (SEQ ID NO:17). In embodiments, any one or more of the listed peptides may be excluded.

In embodiments, the peptide includes a four amino acid residue sequence which, in order, includes a negatively charged (−) amino acid, a neutral (X) amino acid, and two positively charged (+) amino acids. The four amino acid residue sequence including the negatively charged (−) amino acid, neutral (X) amino acid, and two positively charged (+) amino acids may be referred to as a −X++ peptide. In embodiments, the peptide does not have an additional amino acid branch flanking the −X++ peptide (e.g., within 1, 2, 3, 4, 5, 6, 7, 8, 9, or 10 amino acids of the peptide) on the C-terminal side and/or the N-terminal side, which interrupts the exposure of the −X++ sequence to a chelating target.

The peptide may chelate compounds (e.g. transition metals) that are toxic to cells. In embodiments, the portion of the peptide including the −X++ sequence chelates compounds (e.g. transition metals). Thus, in embodiments, the peptide does not include additional C-terminal or N-terminal residues which disrupts contact of the functional peptide sequence to a target (e.g. transition metals). In embodiments, the functional sequence is the amino acid sequence of SEQ ID NO:1.

In embodiments, the peptide is about at least 4 amino acids (e.g., at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 amino acids) in length. In embodiments, the peptide is about at least 4 amino acids in length. In embodiments, the peptide is about at least 5 amino acids in length. In embodiments, the peptide is about at least 6 amino acids in length. In embodiments, the peptide is about at least 7 amino acids in length. In embodiments, the peptide is about at least 8 amino acids in length. In embodiments, the peptide is about at least 9 amino acids in length. In embodiments, the peptide is about at least 10 amino acids in length. In embodiments, the peptide is about at least 11 amino acids in length. In embodiments, the peptide is about at least 12 amino acids in length. In embodiments, the peptide is about at least 13 amino acids in length. In embodiments, the peptide is about at least 14 amino acids in length. In embodiments, the peptide is about at least 15 amino acids in length. In embodiments, the peptide is about at least 16 amino acids in length. In embodiments, the peptide is about at least 17 amino acids in length. In embodiments, the peptide is about at least 18 amino acids in length. In embodiments, the peptide is about at least 19 amino acids in length. In embodiments, the peptide is about at least 20 amino acids in length. In embodiments, the peptide is about at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 amino acids in length.

In embodiments, the peptide is 4 amino acids in length. In embodiments, the peptide is 5 amino acids in length. In embodiments, the peptide is 6 amino acids in length. In embodiments, the peptide is 7 amino acids in length. In embodiments, the peptide is 8 amino acids in length. In embodiments, the peptide is 9 amino acids in length. In embodiments, the peptide is 10 amino acids in length. In embodiments, the peptide is 11 amino acids in length. In embodiments, the peptide is 12 amino acids in length. In embodiments, the peptide is 13 amino acids in length. In embodiments, the peptide is 14 amino acids in length. In embodiments, the peptide is 15 amino acids in length. In embodiments, the peptide is 16 amino acids in length. In embodiments, the peptide is 17 amino acids in length. In embodiments, the peptide is 18 amino acids in length. In embodiments, the peptide is 19 amino acids in length. In embodiments, the peptide is 20 amino acids in length.

In embodiments, the peptide is between about 4 to about 100 amino acids in length. In embodiments, the peptide is between about 8 to about 100 amino acids in length. In embodiments, the peptide is between about 12 to about 100 amino acids in length. In embodiments, the peptide is between about 16 to about 100 amino acids in length. In embodiments, the peptide is between about 20 to about 100 amino acids in length. In embodiments, the peptide is between about 24 to about 100 amino acids in length. In embodiments, the peptide is between about 28 to about 100 amino acids in length. In embodiments, the peptide is between about 32 to about 100 amino acids in length. In embodiments, the peptide is between about 36 to about 100 amino acids in length. In embodiments, the peptide is between about 40 to about 100 amino acids in length. In embodiments, the peptide is between about 44 to about 100 amino acids in length. In embodiments, the peptide is between about 48 to about 100 amino acids in length. In embodiments, the peptide is between about 52 to about 100 amino acids in length. In embodiments, the peptide is between about 56 to about 100 amino acids in length. In embodiments, the peptide is between about 60 to about 100 amino acids in length. In embodiments, the peptide is between about 64 to about 100 amino acids in length. In embodiments, the peptide is between about 68 to about 100 amino acids in length. In embodiments, the peptide is between about 72 to about 100 amino acids in length. In embodiments, the peptide is between about 76 to about 100 amino acids in length. In embodiments, the peptide is between about 80 to about 100 amino acids in length. In embodiments, the peptide is between about 84 to about 100 amino acids in length. In embodiments, the peptide is between about 88 to about 100 amino acids in length. In embodiments, the peptide is between about 92 to about 100 amino acids in length. In embodiments, the peptide is between about 96 to about 100 amino acids in length.

In embodiments, the peptide is between about 4 to about 96 amino acids in length. In embodiments, the peptide is between about 4 to about 92 amino acids in length. In embodiments, the peptide is between about 4 to about 88 amino acids in length. In embodiments, the peptide is between about 4 to about 84 amino acids in length. In embodiments, the peptide is between about 4 to about 80 amino acids in length. In embodiments, the peptide is between about 4 to about 76 amino acids in length. In embodiments, the peptide is between about 4 to about 72 amino acids in length. In embodiments, the peptide is between about 4 to about 68 amino acids in length. In embodiments, the peptide is between about 4 to about 64 amino acids in length. In embodiments, the peptide is between about 4 to about 60 amino acids in length. In embodiments, the peptide is between about 4 to about 56 amino acids in length. In embodiments, the peptide is between about 4 to about 52 amino acids in length. In embodiments, the peptide is between about 4 to about 48 amino acids in length. In embodiments, the peptide is between about 4 to about 44 amino acids in length. In embodiments, the peptide is between about 4 to about 40 amino acids in length. In embodiments, the peptide is between about 4 to about 36 amino acids in length. In embodiments, the peptide is between about 4 to about 32 amino acids in length. In embodiments, the peptide is between about 4 to about 28 amino acids in length. In embodiments, the peptide is between about 4 to about 24 amino acids in length. In embodiments, the peptide is between about 4 to about 20 amino acids in length. In embodiments, the peptide is between about 4 to about 16 amino acids in length. In embodiments, the peptide is between about 4 to about 12 amino acids in length. In embodiments, the peptide is between about 4 to about 8 amino acids in length. In embodiments, the peptide is about 4 amino acids, about 8 amino acids, about 12 amino acids, about 16 amino acids, about 20 amino acids, about 24 amino acids, about 28 amino acids, about 32 amino acids, about 36 amino acids, about 40 amino acids, about 44 amino acids, about 48 amino acids, about 52 amino acids, about 56 amino acids, about 60 amino acids, about 64 amino acids, about 68 amino acids, about 72 amino acids, about 76 amino acids, about 80 amino acids, about 84 amino acids, about 88 amino acids, about 92 amino acids, about 96 amino acids, or about 100 amino acids in length. The peptide length may be any value or subrange within the recited ranges, including endpoints.

In embodiments, the peptide includes an amino acid sequence of SEQ ID NO: 1. In embodiments, the peptide is the amino acid sequence of SEQ ID NO: 1. In embodiments, the peptide includes an amino acid sequence of SEQ ID NO: 2. In embodiments, the peptide is the amino acid sequence of SEQ ID NO: 2. In embodiments, the peptide includes an amino acid sequence of SEQ ID NO: 6. In embodiments, the peptide is the amino acid sequence of SEQ ID NO: 6. In embodiments, the peptide includes an amino acid sequence of SEQ ID NO: 7. In embodiments, the peptide is the amino acid sequence of SEQ ID NO: 7. In embodiments, the peptide includes an amino acid sequence of SEQ ID NO: 8. In embodiments, the peptide is the amino acid sequence of SEQ ID NO: 8. In embodiments, the peptide includes an amino acid sequence of SEQ ID NO: 9. In embodiments, the peptide is the amino acid sequence of SEQ ID NO: 9. In embodiments, the peptide includes an amino acid sequence of SEQ ID NO: 10. In embodiments, the peptide is the amino acid sequence of SEQ ID NO: 10. In embodiments, the peptide includes an amino acid sequence of SEQ ID NO: 11. In embodiments, the peptide is the amino acid sequence of SEQ ID NO: 11. In embodiments, the peptide includes an amino acid sequence of SEQ ID NO: 12. In embodiments, the peptide is the amino acid sequence of SEQ ID NO: 12. In embodiments, the peptide includes an amino acid sequence of SEQ ID NO: 13. In embodiments, the peptide is the amino acid sequence of SEQ ID NO: 13. In embodiments, the peptide includes an amino acid sequence of SEQ ID NO: 14. In embodiments, the peptide is the amino acid sequence of SEQ ID NO: 14. In embodiments, the peptide includes an amino acid sequence of SEQ ID NO: 15. In embodiments, the peptide is the amino acid sequence of SEQ ID NO: 15. In embodiments, the peptide includes an amino acid sequence of SEQ ID NO: 16. In embodiments, the peptide is the amino acid sequence of SEQ ID NO: 16. In embodiments, the peptide includes an amino acid sequence of SEQ ID NO: 17. In embodiments, the peptide is the amino acid sequence of SEQ ID NO: 17. In embodiments, the sequence of any one or more of the listed peptides may be excluded.

In embodiments, the cell culture medium is serum free. In embodiments, the cell culture medium is albumin-free. In embodiments, the cell culture medium is serum free and albumin-free. In embodiments, the cell culture medium is substantially serum free. In embodiments, the cell culture medium is substantially albumin-free. In embodiments, the cell culture medium is substantially serum free and substantially albumin-free.

In embodiments, the cell culture supplement provided herein including embodiments thereof is provided as a 1× to 100× concentration. In embodiments, the cell culture supplement provided herein including embodiments thereof is provided as a 1×, 2×, 3×, 5×, 10×, 20×, 25×, 50×, 75× or 100× concentration. In embodiments, the cell culture supplement provided herein including embodiments thereof is provided as a 1× concentration. In embodiments, the cell culture supplement provided herein including embodiments thereof is provided as a 2λ concentration. In embodiments, the cell culture supplement provided herein including embodiments thereof is provided as a 3× concentration. In embodiments, the cell culture supplement provided herein including embodiments thereof is provided as a 5× concentration. In embodiments, the cell culture supplement provided herein including embodiments thereof is provided as a 10× concentration. In embodiments, the cell culture supplement provided herein including embodiments thereof is provided as a 20× concentration. In embodiments, the cell culture supplement provided herein including embodiments thereof is provided as a 25× concentration. In embodiments, the cell culture supplement provided herein including embodiments thereof is provided as a 50× concentration. In embodiments, the cell culture supplement provided herein including embodiments thereof is provided as a 75× concentration. In embodiments, the cell culture supplement provided herein including embodiments thereof is provided as a 100× concentration.

In embodiments, the cell culture medium or cell culture supplement provided herein further includes a superoxide scavenger. In embodiments, the superoxide scavenger includes a compound containing (2,2,6,6-tertramethyl-1-yl)oxyl or variants thereof, or a flavonoid. In embodiments, the superoxide scavenger includes (2,2,6,6-tertramethyl-1-yl)oxyl. In embodiments, the superoxide scavenger includes one or more variants of (2,2,6,6-tertramethyl-1-yl)oxyl. In embodiments, the superoxide scavenger includes a flavonoid. In embodiments, one or more of the recited superoxide scavengers may be expressly excluded.

In embodiments, the superoxide scavenger includes a compound that is not native (endogenous) to cells (e.g., the cells being cultured). In embodiments, the superoxide scavenger may include TEMPO (2,2,6,6-tetramethylpiperidine-N-oxyl), hydroxy-TEMPO (4-hydroxy-2,2,6,6-tetramethyl-piperidine-N-oxyl), TEMPOL (1-oxyl-2,2,6,6-tetramethyl-4-hydroxypiperidine), or a variant thereof. In embodiments, the superoxide scavenger includes TEMPO. In embodiments, the superoxide scavenger includes a variant of TEMPO. In embodiments, the superoxide scavenger includes TEMPOL. In embodiments, the superoxide scavenger includes a variant of TEMPOL. In embodiments, the superoxide scavenger includes Mito-TEMPO ((2-(2,2,6,6-tetramethylpiperidin-1-oxyl-4-ylamino)-2-oxoethyl)triphenylphosphonium chloride). Additional superoxide scavengers can be found, for example, in U.S. Pat. No. 6,759,430, which is incorporated herein by reference for all that is disclosed, including compounds, compositions, methods, molecules, synthesis, etc. In embodiments, one or more of the recited superoxide scavengers may be expressly excluded.

The concentration of the superoxide scavenger may refer to the concentration of the superoxide scavenger in a cell culture medium provided herein including embodiments thereof. The concentration of the superoxide scavenger may refer to the final concentration in a complete cell culture medium (i.e. basal medium with a cell culture supplement provided herein added). In embodiments, the concentration of the superoxide scavenger is between about 1 μM to about 25 μM. In embodiments, the concentration of the superoxide scavenger is between about 2 μM to about 25 μM. In embodiments, the concentration of the superoxide scavenger is between about 3 μM to about 25 μM. In embodiments, the concentration of the superoxide scavenger is between about 4 μM to about 25 μM. In embodiments, the concentration of the superoxide scavenger is between about 5 μM to about 25 μM. In embodiments, the concentration of the superoxide scavenger is between about 6 μM to about 25 μM. In embodiments, the concentration of the superoxide scavenger is between about 7 μM to about 25 μM. In embodiments, the concentration of the superoxide scavenger is between about 8 μM to about 25 μM. In embodiments, the concentration of the superoxide scavenger is between about 9 μM to about 25 μM. In embodiments, the concentration of the superoxide scavenger is between about 10 μM to about 25 μM. In embodiments, the concentration of the superoxide scavenger is between about 11 μM to about 25 μM. In embodiments, the concentration of the superoxide scavenger is between about 12 μM to about 25 μM. In embodiments, the concentration of the superoxide scavenger is between about 13 μM to about 25 μM. In embodiments, the concentration of the superoxide scavenger is between about 14 μM to about 25 μM. In embodiments, the concentration of the superoxide scavenger is between about 15 μM to about 25 μM. In embodiments, the concentration of the superoxide scavenger is between about 16 μM to about 25 μM. In embodiments, the concentration of the superoxide scavenger is between about 17 μM to about 25 μM. In embodiments, the concentration of the superoxide scavenger is between about 18 μM to about 25 μM. In embodiments, the concentration of the superoxide scavenger is between about 19 μM to about 25 μM. In embodiments, the concentration of the superoxide scavenger is between about 20 μM to about 25 μM. In embodiments, the concentration of the superoxide scavenger is between about 21 μM to about 25 μM. In embodiments, the concentration of the superoxide scavenger is between about 22 μM to about 25 μM. In embodiments, the concentration of the superoxide scavenger is between about 23 μM to about 25 μM. In embodiments, the concentration of the superoxide scavenger is between about 24 μM to about 25 μM. The concentration may be any value or subrange within any range recited herein, including endpoints.

In embodiments, the concentration of the superoxide scavenger is between about 1 μM to about 24 μM. In embodiments, the concentration of the superoxide scavenger is between about 1 μM to about 23 μM. In embodiments, the concentration of the superoxide scavenger is between about 1 μM to about 22 μM. In embodiments, the concentration of the superoxide scavenger is between about 1 μM to about 21 μM. In embodiments, the concentration of the superoxide scavenger is between about 1 μM to about 20 μM. In embodiments, the concentration of the superoxide scavenger is between about 1 μM to about 19 μM. In embodiments, the concentration of the superoxide scavenger is between about 1 μM to about 18 μM. In embodiments, the concentration of the superoxide scavenger is between about 1 μM to about 17 μM. In embodiments, the concentration of the superoxide scavenger is between about 16 μM to about 24 μM. In embodiments, the concentration of the superoxide scavenger is between about 15 μM to about 24 μM. In embodiments, the concentration of the superoxide scavenger is between about 14 μM to about 24 μM. In embodiments, the concentration of the superoxide scavenger is between about 13 μM to about 24 μM. In embodiments, the concentration of the superoxide scavenger is between about 12 μM to about 24 μM. In embodiments, the concentration of the superoxide scavenger is between about 11 μM to about 24 μM. In embodiments, the concentration of the superoxide scavenger is between about 10 μM to about 24 μM. In embodiments, the concentration of the superoxide scavenger is between about 9 μM to about 24 μM. In embodiments, the concentration of the superoxide scavenger is between about 8 μM to about 24 μM. In embodiments, the concentration of the superoxide scavenger is between about 7 μM to about 24 μM. In embodiments, the concentration of the superoxide scavenger is between about 6 μM to about 24 μM. In embodiments, the concentration of the superoxide scavenger is between about 5 μM to about 24 μM. In embodiments, the concentration of the superoxide scavenger is between about 4 μM to about 24 μM. In embodiments, the concentration of the superoxide scavenger is between about 3 μM to about 24 μM. In embodiments, the concentration of the superoxide scavenger is between about 2 μM to about 24 μM. In embodiments, the concentration of the superoxide scavenger is about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, or about 25 μM. The concentration of the superoxide scavenger provided herein including embodiments thereof may be any value or subrange within the recited ranges, including endpoints.

In embodiments, the final concentration of the superoxide scavenger medium provided herein is between about 5 to about 25 μM. The final concentration of the superoxide scavenger may refer to the superoxide scavenger concentration in a complete cell culture medium (i.e. basal medium with a cell culture supplement provided herein added). In embodiments, the final concentration of the superoxide scavenger medium provided herein is between about 6 to about 25 μM. In embodiments, the final concentration of the superoxide scavenger medium provided herein is between about 7 to about 25 μM. In embodiments, the final concentration of the superoxide scavenger medium provided herein is between about 8 to about 25 μM. In embodiments, the final concentration of the superoxide scavenger medium provided herein is between about 9 to about 25 μM. In embodiments, the final concentration of the superoxide scavenger medium provided herein is between about 10 to about 25 μM. In embodiments, the final concentration of the superoxide scavenger medium provided herein is between about 11 to about 25 μM. In embodiments, the final concentration of the superoxide scavenger medium provided herein is between about 12 to about 25 μM. In embodiments, the final concentration of the superoxide scavenger medium provided herein is between about 13 to about 25 μM. In embodiments, the final concentration of the superoxide scavenger medium provided herein is between about 14 to about 25 μM. In embodiments, the final concentration of the superoxide scavenger medium provided herein is between about 15 to about 25 μM. In embodiments, the final concentration of the superoxide scavenger medium provided herein is between about 16 to about 25 μM. In embodiments, the final concentration of the superoxide scavenger medium provided herein is between about 17 to about 25 μM. In embodiments, the final concentration of the superoxide scavenger medium provided herein is between about 18 to about 25 μM. In embodiments, the final concentration of the superoxide scavenger medium provided herein is between about 19 to about 25 μM. In embodiments, the final concentration of the superoxide scavenger medium provided herein is between about 20 to about 25 μM. In embodiments, the final concentration of the superoxide scavenger medium provided herein is between about 21 to about 25 μM. In embodiments, the final concentration of the superoxide scavenger medium provided herein is between about 22 to about 25 μM. In embodiments, the final concentration of the superoxide scavenger medium provided herein is between about 23 to about 25 μM. In embodiments, the final concentration of the superoxide scavenger medium provided herein is between about 24 to about 25 μM.

In embodiments, the final concentration of the superoxide scavenger medium provided herein is between about 5 to about 24 μM. In embodiments, the final concentration of the superoxide scavenger medium provided herein is between about 5 to about 23 μM. In embodiments, the final concentration of the superoxide scavenger medium provided herein is between about 5 to about 22 μM. In embodiments, the final concentration of the superoxide scavenger medium provided herein is between about 5 to about 21 μM. In embodiments, the final concentration of the superoxide scavenger medium provided herein is between about 5 to about 20 μM. In embodiments, the final concentration of the superoxide scavenger medium provided herein is between about 5 to about 19 μM. In embodiments, the final concentration of the superoxide scavenger medium provided herein is between about 5 to about 18 μM. In embodiments, the final concentration of the superoxide scavenger medium provided herein is between about 5 to about 17 μM. In embodiments, the final concentration of the superoxide scavenger medium provided herein is between about 5 to about 16 μM. In embodiments, the final concentration of the superoxide scavenger medium provided herein is between about 5 to about 15 μM. In embodiments, the final concentration of the superoxide scavenger medium provided herein is between about 5 to about 14 μM. In embodiments, the final concentration of the superoxide scavenger medium provided herein is between about 5 to about 13 μM. In embodiments, the final concentration of the superoxide scavenger medium provided herein is between about 5 to about 12 μM. In embodiments, the final concentration of the superoxide scavenger medium provided herein is between about 5 to about 11 μM. In embodiments, the final concentration of the superoxide scavenger medium provided herein is between about 5 to about 10 μM. In embodiments, the final concentration of the superoxide scavenger medium provided herein is between about 5 to about 9 μM. In embodiments, the final concentration of the superoxide scavenger medium provided herein is between about 5 to about 8 μM. In embodiments, the final concentration of the superoxide scavenger medium provided herein is between about 5 to about 7 μM. In embodiments, the final concentration of the superoxide scavenger medium provided herein is between about 5 to about 6 μM. In embodiments, the final concentration of the superoxide scavenger medium provided herein is about 5, 6, 7, 8, 9, 10, 11, 12, 13, 14, 15, 16, 17, 18, 19, 20, 21, 22, 23, 24 or 25 μM. The final concentration of the superoxide scavenger medium may be provided at a concentration encompassing any value or subrange within any range recited herein, including endpoints.

In embodiments, the cell culture medium or cell culture supplement provided herein includes a vitamin E or an analog thereof. In embodiments, the vitamin E analog includes a 6-chromanol group moiety.

In embodiments, the vitamin E or analog or variant thereof includes α-tocopherol, β-tocopherol, γ-tocopherol, δ-tocopherol, α-tocotrienol, β-tocotrienol, δ-tocotrienol, γ-tocotrienol, α-tocoperol succinate, α-tocopherol monoglucoside, γ-tocopherol N,N-dimethyl glycine ester, or a substitution, isoform pure, racemic mixture, and/or mixture thereof.

In embodiments the vitamin E or analog or variant thereof includes α-tocopherol. In embodiments the vitamin E or analog or variant thereof includes β-tocopherol. In embodiments the vitamin E or analog or variant thereof includes γ-tocopherol. In embodiments the vitamin E or analog or variant thereof includes δ-tocopherol. In embodiments the vitamin E or analog or variant thereof includes α-tocotrienol. In embodiments the vitamin E or analog or variant thereof includes β-tocotrienol. In embodiments the vitamin E or analog or variant thereof includes δ-tocotrienol. In embodiments the vitamin E or analog or variant thereof includes γ-tocotrienol. In embodiments the vitamin E or analog or variant thereof includes α-tocoperol succinate. In embodiments the vitamin E or analog or variant thereof includes α-tocopherol monoglucoside. In embodiments the vitamin E or analog or variant thereof includes γ-tocopherol N,N-dimethyl glycine ester. In embodiments, the vitamin E or analog or variant thereof includes endogenous metabolites of vitamin E. In embodiments, the vitamin E or analog or variant thereof includes alpha-tocopherol hydroquinone. In embodiments, the vitamin E or analog or variant thereof includes endogenous metabolites of vitamin E, alpha-tocopherol hydroquinone, and the like.

In embodiments, the vitamin E or analog or variant thereof includes a pure isoform of a vitamin E or analog or variant provided herein. In embodiments, the vitamin E or analog or variant thereof includes a substituted vitamin E or analog or variant provided herein. In embodiments, the vitamin E or analog or variant thereof includes a racemic mixture of a vitamin E or analog or variant provided herein. In embodiments, the vitamin E or analog or variant thereof includes a mixture of two or more vitamin E compounds or analogs or variants provided herein. In embodiments, one or more of the recited vitamin E compounds may be expressly excluded.

The concentration of Vitamin E or an analog thereof may refer to the concentration in a cell culture medium provided herein, including embodiments thereof. The concentration of Vitamin E or an analog thereof may refer to the final concentration of said Vitamin E or analog in a complete culture medium (i.e. basal medium with a cell culture supplement provided herein added). Thus, in embodiments, the concentration of Vitamin E or an analog thereof is between about 0.1 μg/ml to about 4.5 μg/ml. In embodiments, the concentration of Vitamin E or an analog thereof is between about 0.5 μg/ml to about 4.5 μg/ml. In embodiments, the concentration of Vitamin E or an analog thereof is between about 1 μg/ml to about 4.5 μg/ml. In embodiments, the concentration of Vitamin E or an analog thereof is between about 1.5 μg/ml to about 4.5 μg/ml. In embodiments, the concentration of Vitamin E or an analog thereof is between about 2 μg/ml to about 4.5 μg/ml. In embodiments, the concentration of Vitamin E or an analog thereof is between about 2.5 μg/ml to about 4.5 μg/ml. In embodiments, the concentration of Vitamin E or an analog thereof is between about 3 μg/ml to about 4.5 μg/ml. In embodiments, the concentration of Vitamin E or an analog thereof is between about 3.5 μg/ml to about 4.5 μg/ml. In embodiments, the concentration of Vitamin E or an analog thereof is between about 4 μg/ml to about 4.5 μg/ml.

In embodiments, the concentration of Vitamin E or an analog thereof is between about 0.1 μg/ml to about 4 μg/ml. In embodiments, the concentration of Vitamin E or an analog thereof is between about 0.1 μg/ml to about 3.5 μg/ml. In embodiments, the concentration of Vitamin E or an analog thereof is between about 0.1 μg/ml to about 3 μg/ml. In embodiments, the concentration of Vitamin E or an analog thereof is between about 0.1 μg/ml to about 2.5 μg/ml. In embodiments, the concentration of Vitamin E or an analog thereof is between about 0.1 μg/ml to about 2 μg/ml. In embodiments, the concentration of Vitamin E or an analog thereof is between about 0.1 μg/ml to about 1.5 μg/ml. In embodiments, the concentration of Vitamin E or an analog thereof is between about 0.1 μg/ml to about 1 μg/ml. In embodiments, the concentration of Vitamin E or an analog thereof is between about 0.1 μg/ml to about 0.5 μg/ml. In embodiments, the concentration of Vitamin E or an analog thereof is about 0.1 μg/ml, about 0.5 μg/ml, about 1 μg/ml, about 1.5 μg/ml, about 2 μg/ml, about 2.5 μg/ml, about 3 μg/ml, about 3.5 μg/ml, about 4 μg/ml, or about 4.5 μg/ml. The concentration of Vitamin E or an analog thereof may be any value or subrange within any range recited herein, including endpoints.

In embodiments, the concentration of Vitamin E is 0.5 μg/ml, 0.6 μg/ml, 0.7 μg/ml, 0.8 μg/ml, 0.9 μg/ml, 1.0 μg/ml, 1.1 μg/ml, 1.2 μg/ml, 1.3 μg/ml, 1.4 μg/ml, 1.5 μg/ml, 1.6 μg/ml, 1.7 μg/ml, 1.8 μg/ml, 1.9 μg/ml, 2.0 μg/ml, 2.1 μg/ml, 2.2 μg/ml, 2.3 μg/ml, 2.4 μg/ml, 2.5 μg/ml, 2.6 μg/ml, 2.7 μg/ml, 2.8 μg/ml, 2.9 μg/ml, 3 μg/ml, 3.1 μg/ml, 3.2 μg/ml, 3.3 μg/ml, 3.4 μg/ml, 3.5 μg/ml, 3.6 μg/ml, 3.7 μg/ml, 3.8 μg/ml, 3.9 μg/ml, 4 μg/ml, 4.1 μg/ml, 4.2 μg/ml, 4.3 μg/ml, 4.4 μg/ml, 4.5 μg/ml, 4.6 μg/ml, 4.7 μg/ml, 4.8 μg/ml, 4.9 μg/ml, or 5.0 μg/ml. The concentration of Vitamin E or an analog thereof may be any value or subrange within the recited ranges, including endpoints.

In embodiments, the concentration of a Vitamin E analog is between about 2 to about 100 μM. For example in some embodiments, the cell culture medium or the cell culture supplement provides a final concentration of a Vitamin E analog of about 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 51 μM, 52 μM, 53 μM, 54 μM, 55 μM, 56 μM, 57 μM, 58 μM, 59 μM, 60 μM, 61 μM, 62 μM, 63 μM, 64 μM, 65 μM, 66 μM, 67 μM, 68 μM, 69 μM, 70 μM, 71 μM, 72 μM, 73 μM, 74 μM, 75 μM, 76 μM, 77 μM, 78 μM, 79 μM, 80 μM, or more, or any amount in between. The concentration of the Vitamin E analog may be any value or subrange within the recited ranges, including endpoints.

In embodiments, the vitamin E analog is water soluble. In embodiments, the vitamin E analog includes 6-hydroxy-2,5,7,8-tetramethylchroman-2-carboxylic acid (Trolox).

The concentration of Trolox may refer to the concentration in a cell culture medium as provided herein, including embodiments thereof. The concentration of Trolox may refer to the concentration in a complete culture medium (i.e. basal medium with a cell culture supplement provided herein added). In embodiments, the concentration of Trolox is at least about 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 51 μM, 52 μM, 53 μM, 54 μM, 55 μM, 56 μM, 57 μM, 58 μM, 59 μM, 60 μM, 61 μM, 62 μM, 63 μM, 64 μM, 65 μM, 66 μM, 67 μM, 68 μM, 69 μM, 70 μM, 71 μM, 72 μM, 73 μM, 74 μM, 75 μM, 76 μM, 77 μM, 78 μM, 79 μM, 80 μM, or more. The concentration of Trolox may be any value or subrange within the recited ranges, including endpoints.

In embodiments, the cell culture medium or cell culture supplement provided herein further includes a hydrogen peroxide reducing reagent. In embodiments, the hydrogen peroxide reducing agent is cell permeable. In embodiments, the hydrogen peroxide reducing agent is not cell permeable. In embodiments, the hydrogen peroxide reducing agent is a mixture of cell permeable and not cell permeable reagents. In embodiments, the hydrogen peroxide reducing agent includes glutathione, N-acetyl cysteine, cysteine, sodium selenite, mannitol, a flavonoid, lipoic acid, or any combination thereof. In embodiments, the hydrogen peroxide reducing agent includes glutathione. In embodiments, the hydrogen peroxide reducing agent includes N-acetyl cysteine. In embodiments, the hydrogen peroxide reducing agent includes cysteine. In embodiments, the hydrogen peroxide reducing agent includes sodium selenite. In embodiments, the hydrogen peroxide reducing agent includes mannitol. In embodiments, the hydrogen peroxide reducing agent includes a flavonoid. In embodiments, the hydrogen peroxide reducing agent includes lipoic acid. In embodiments, the hydrogen peroxide reducing agent includes a combination of two or more hydrogen peroxide reducing agents provided herein. In embodiments, one or more of the recited hydrogen peroxide reducing reagents may be expressly excluded.

The concentration of hydrogen peroxide reducing agent may refer to the concentration in a cell culture medium provided herein including embodiments thereof. The concentration of hydrogen peroxide reducing agent may refer to the concentration in a complete culture medium (i.e. basal medium with cell culture supplement added).

Thus, in embodiments, the concentration of glutathione is between about 1 μg/ml to about 200 μg/ml. In embodiments, the concentration of glutathione is between about 20 μg/ml to about 200 μg/ml. In embodiments, the concentration of glutathione is between about 40 μg/ml to about 200 μg/ml. In embodiments, the concentration of glutathione is between about 60 μg/ml to about 200 μg/ml. In embodiments, the concentration of glutathione is between about 80 μg/ml to about 200 μg/ml. In embodiments, the concentration of glutathione is between about 100 μg/ml to about 200 μg/ml. In embodiments, the concentration of glutathione is between about 120 μg/ml to about 200 μg/ml. In embodiments, the concentration of glutathione is between about 140 μg/ml to about 180 μg/ml. In embodiments, the concentration of glutathione is between about 160 μg/ml to about 200 μg/ml. In embodiments, the concentration of glutathione is between about 180 μg/ml to about 200 μg/ml.

In embodiments, the concentration of glutathione is between about 1 μg/ml to about 200 μg/ml. In embodiments, the concentration of glutathione is between about 1 μg/ml to about 180 μg/ml. In embodiments, the concentration of glutathione is between about 1 μg/ml to about 160 μg/ml. In embodiments, the concentration of glutathione is between about 1 μg/ml to about 140 μg/ml. In embodiments, the concentration of glutathione is between about 1 μg/ml to about 120 μg/ml. In embodiments, the concentration of glutathione is between about 1 μg/ml to about 100 μg/ml. In embodiments, the concentration of glutathione is between about 1 μg/ml to about 80 μg/ml. In embodiments, the concentration of glutathione is between about 1 μg/ml to about 60 μg/ml. In embodiments, the concentration of glutathione is between about 1 μg/ml to about 40 μg/ml. In embodiments, the concentration of glutathione is between about 1 μg/ml to about 20 μg/ml. In embodiments, the concentration of glutathione is about 1, 20, 40, 60, 80, 100, 120, 140, 160, 180 or 200 μg/ml. The concentration of glutathione may be any value or subrange within the recited ranges, including endpoints.

In embodiments, the cell culture medium or cell culture supplement provides a final concentration of glutathione in the complete culture medium (i.e., basal medium with supplement added) of about 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, 20 μg/ml, 21 μg/ml, 22 μg/ml, 23 μg/ml, 24 μg/ml, 25 μg/ml, 26 μg/ml, 27 μg/ml, 28 μg/ml, 29 μg/ml, 30 μg/ml, 31 μg/ml, 32 μg/ml, 33 μg/ml, 34 μg/ml, 35 μg/ml, 36 μg/ml, 37 μg/ml, 38 μg/ml, 39 μg/ml, 40 μg/ml, 41 μg/ml, 42 μg/ml, 43 μg/ml, 44 μg/ml, 45 μg/ml, 46 μg/ml, 47 μg/ml, 48 μg/ml, 49 μg/ml, 50 μg/ml, 51 μg/ml, 52 μg/ml, 53 μg/ml, 54 μg/ml, 55 μg/ml, 56 μg/ml, 57 μg/ml, 58 μg/ml, 59 μg/ml, 60 μg/ml, 61 μg/ml, 62 μg/ml, 63 μg/ml, 64 μg/ml, 65 μg/ml, 66 μg/ml, 67 μg/ml, 68 μg/ml, 69 μg/ml, 70 μg/ml, 71 μg/ml, 72 μg/ml, 73 μg/ml, 74 μg/ml, 75 μg/ml, 76 μg/ml, 77 μg/ml, 78 μg/ml, 79 μg/ml, 80 μg/ml, 81 μg/ml, 82 μg/ml, 83 μg/ml, 84 μg/ml, 85 μg/ml, 86 μg/ml, 87 μg/ml, 88 μg/ml, 89 μg/ml, 90 μg/ml, 91 μg/ml, 92 μg/ml, 93 μg/ml, 94 μg/ml, 95 μg/ml, 96 μg/ml, 97 μg/ml, 98 μg/ml, 99 μg/ml, 100 μg/ml, 105 μg/ml, 110 μg/ml, 115 μg/ml, 120 μg/ml, 125 μg/ml, 130 μg/ml, 135 μg/ml, 140 μg/ml, 145 μg/ml, or 150 μg/ml. The final concentration of glutathione may be any value or subrange within the recited ranges, including endpoints.

In embodiments, the concentration of lipoic acid is between about 0.05 μg/ml to about 12 μg/ml. In embodiments, the concentration of lipoic acid is between about 0.1 μg/ml to about 12 μg/ml. In embodiments, the concentration of lipoic acid is between about 0.5 μg/ml to about 12 μg/ml. In embodiments, the concentration of lipoic acid is between about 1 μg/ml to about 12 μg/ml. In embodiments, the concentration of lipoic acid is between about 1.5 μg/ml to about 12 μg/ml. In embodiments, the concentration of lipoic acid is between about 2 μg/ml to about 12 μg/ml. In embodiments, the concentration of lipoic acid is between about 2.5 μg/ml to about 12 μg/ml. In embodiments, the concentration of lipoic acid is between about 3 μg/ml to about 12 μg/ml. In embodiments, the concentration of lipoic acid is between about 3.5 μg/ml to about 12 μg/ml. In embodiments, the concentration of lipoic acid is between about 4 μg/ml to about 12 μg/ml. In embodiments, the concentration of lipoic acid is between about 4.5 μg/ml to about 12 μg/ml. In embodiments, the concentration of lipoic acid is between about 5 μg/ml to about 12 μg/ml. In embodiments, the concentration of lipoic acid is between about 5.5 μg/ml to about 12 μg/ml. In embodiments, the concentration of lipoic acid is between about 6 μg/ml to about 12 μg/ml. In embodiments, the concentration of lipoic acid is between about 6.5 μg/ml to about 12 μg/ml. In embodiments, the concentration of lipoic acid is between about 7 μg/ml to about 12 μg/ml. In embodiments, the concentration of lipoic acid is between about 7.5 μg/ml to about 12 μg/ml. In embodiments, the concentration of lipoic acid is between about 8 μg/ml to about 12 μg/ml. In embodiments, the concentration of lipoic acid is between about 8.5 μg/ml to about 12 μg/ml. In embodiments, the concentration of lipoic acid is between about 9 μg/ml to about 12 μg/ml. In embodiments, the concentration of lipoic acid is between about 9.5 μg/ml to about 12 μg/ml. In embodiments, the concentration of lipoic acid is between about 10 μg/ml to about 12 μg/ml. In embodiments, the concentration of lipoic acid is between about 10.5 μg/ml to about 12 μg/ml. In embodiments, the concentration of lipoic acid is between about 11 μg/ml to about 12 μg/ml. In embodiments, the concentration of lipoic acid is between about 11.5 μg/ml to about 12 μg/ml.

In embodiments, the concentration of lipoic acid is between about 0.05 μg/ml to about 11.5 μg/ml. In embodiments, the concentration of lipoic acid is between about 0.05 μg/ml to about 11 μg/ml. In embodiments, the concentration of lipoic acid is between about 0.05 μg/ml to about 10.5 μg/ml. In embodiments, the concentration of lipoic acid is between about 0.05 ng/ml to about 10 μg/ml. In embodiments, the concentration of lipoic acid is between about 0.05 ng/ml to about 9.5 μg/ml. In embodiments, the concentration of lipoic acid is between about 0.05 ng/ml to about 9 μg/ml. In embodiments, the concentration of lipoic acid is between about 0.05 ng/ml to about 8.5 μg/ml. In embodiments, the concentration of lipoic acid is between about 0.1 ng/ml to about 8 μg/ml. In embodiments, the concentration of lipoic acid is between about 0.05 ng/ml to about 7.5 μg/ml. In embodiments, the concentration of lipoic acid is between about 0.05 μg/ml to about 7 μg/ml. In embodiments, the concentration of lipoic acid is between about 0.05 μg/ml to about 6.5 μg/ml. In embodiments, the concentration of lipoic acid is between about 0.05 μg/ml to about 6 μg/ml. In embodiments, the concentration of lipoic acid is between about 0.05 μg/ml to about 5.5 μg/ml. In embodiments, the concentration of lipoic acid is between about 0.05 μg/ml to about 4 μg/ml. In embodiments, the concentration of lipoic acid is between about 0.05 μg/ml to about 3.5 μg/ml. In embodiments, the concentration of lipoic acid is between about 0.05 μg/ml to about 3 μg/ml. In embodiments, the concentration of lipoic acid is between about 0.05 μg/ml to about 2.5 μg/ml. In embodiments, the concentration of lipoic acid is between about 0.05 μg/ml to about 2 μg/ml. In embodiments, the concentration of lipoic acid is between about 0.05 μg/ml to about 1.5 μg/ml. In embodiments, the concentration of lipoic acid is between about 0.05 μg/ml to about 1 μg/ml. In embodiments, the concentration of lipoic acid is between about 0.05 μg/ml to about 0.5 μg/ml. In embodiments, the concentration of lipoic acid is about 0.05 μg/ml, about 0.1 μg/ml, about 0.5 μg/ml, about 1 μg/ml, about 1.5 ng/ml, about 2 μg/ml, about 2.5 μg/ml, about 3 μg/ml, about 3.5 μg/ml, about 4 μg/ml, about 4.5 ng/ml, about 5 μg/ml, about 5.5 μg/ml, about 6 μg/ml, about 6.5 μg/ml, about 7 μg/ml, about 7.5 μg/ml, about 8 μg/ml, about 8.5 μg/ml, about 9 μg/ml, about 9.5 μg/ml, about 10 μg/ml, about 10.5 μg/ml, about 11 μg/ml, about 11.5 μg/ml, or about 12 μg/ml.

In embodiments, the concentration of lipoic acid is about 0.05 μg/ml, 0.075 μg/ml, 0.1 μg/ml, 0.2 μg/ml, 0.3 μg/ml, 0.4 μg/ml, 0.5 μg/ml, 0.6 μg/ml, 0.7 μg/ml, 0.8 μg/ml, 0.9 μg/ml, 1.0 μg/ml, 1.25 μg/ml, 1.50 μg/ml, 1.75 μg/ml, 2.0 μg/ml, 2.25 μg/ml, 2.5 μg/ml, 2.75 μg/ml, 3.0 μg/ml, 3.25 μg/ml, 3.5 μg/ml, 3.75 μg/ml, 4.0 μg/ml, 4.25 μg/ml, 4.5 μg/ml, 4.75 μg/ml, 5.0 μg/ml, 5.25 μg/ml, 5.5 μg/ml, 5.75 μg/ml, 6.0 μg/ml, 6.25 μg/ml, 6.5 μg/ml, 6.75 μg/ml, 7.0 μg/ml, 7.25 μg/ml, 7.5 μg/ml, 7.75 μg/ml, 8.0 μg/ml, 8.25 μg/ml, 8.5 μg/ml, 8.75 μg/ml, 9.0 μg/ml, 9.25 μg/ml, 9.5 μg/ml, 9.75 μg/ml, 10.0 μg/ml, 11.0 μg/ml, 12.0 μg/ml, or more. The concentration of lipoic acid may be any value or subrange within the recited ranges, including endpoints.

In embodiments, the cell culture medium or cell culture supplement provided herein further includes a stabilizer molecule. Stabilizer molecules stabilize proteins against environmental stress, for example oxidative stress. Without wishing to be bound by theory, it is believed that the stabilizer molecule can mitigate lipid peroxidation and fatty acid formation. In embodiments, the stabilizer molecule is a sugar or polyol. In embodiments, the stabilizer molecule is trehalose, mannitol, sucrose, maltose, lactose, sorbitol, or glycerol. In embodiments, the stabilizer molecule is mannitol.

The concentration of a stabilizer may refer to the concentration in a cell culture medium provided herein including embodiments thereof. The concentration of a stabilizer may refer to the concentration in a complete culture medium (i.e. basal medium with a cell culture supplement provided herein added).

In embodiments, the concentration of mannitol is between about 1 mM to about 150 mM. In embodiments, the concentration of mannitol is between about 10 mM to about 150 mM. In embodiments, the concentration of mannitol is between about 20 mM to about 150 mM. In embodiments, the concentration of mannitol is between about 30 mM to about 150 mM. In embodiments, the concentration of mannitol is between about 40 mM to about 150 mM. In embodiments, the concentration of mannitol is between about 50 mM to about 150 mM. In embodiments, the concentration of mannitol is between about 60 mM to about 150 mM. In embodiments, the concentration of mannitol is between about 70 mM to about 150 mM. In embodiments, the concentration of mannitol is between about 80 mM to about 150 mM. In embodiments, the concentration of mannitol is between about 90 mM to about 150 mM. In embodiments, the concentration of mannitol is between about 100 mM to about 150 mM. In embodiments, the concentration of mannitol is between about 110 mM to about 150 mM. In embodiments, the concentration of mannitol is between about 120 mM to about 150 mM. In embodiments, the concentration of mannitol is between about 130 mM to about 150 mM. In embodiments, the concentration of mannitol is between about 140 mM to about 150 mM.

In embodiments, the concentration of mannitol is between about 1 mM to about 140 mM. In embodiments, the concentration of mannitol is between about 1 mM to about 130 mM. In embodiments, the concentration of mannitol is between about 1 mM to about 120 mM. In embodiments, the concentration of mannitol is between about 1 mM to about 110 mM. In embodiments, the concentration of mannitol is between about 1 mM to about 100 mM. In embodiments, the concentration of mannitol is between about 1 mM to about 90 mM. In embodiments, the concentration of mannitol is between about 1 mM to about 80 mM. In embodiments, the concentration of mannitol is between about 1 mM to about 70 mM. In embodiments, the concentration of mannitol is between about 1 mM to about 60 mM. In embodiments, the concentration of mannitol is between about 1 mM to about 50 mM. In embodiments, the concentration of mannitol is between about 1 mM to about 40 mM. In embodiments, the concentration of mannitol is between about 1 mM to about 30 mM. In embodiments, the concentration of mannitol is between about 1 mM to about 20 mM. In embodiments, the concentration of mannitol is between about 1 mM to about 10 mM.

In embodiments, the concentration of mannitol is about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 105 mM, 110 mM, 115 mM, 120 mM, 125 mM, 130 mM, 135 mM, 140 mM, 145 mM, or 150 mM.

In embodiments, the concentration of mannitol is between about 0.05 μg/ml to about 10 μg/ml. The concentration of mannitol may be any value or subrange within the recited ranges, including endpoints.

In embodiments, the cell culture supplement is serum-free. In embodiments, the cell culture supplement is albumin-free. In embodiments, the cell culture supplement is serum-free and albumin-free. In embodiments, the cell culture supplement is substantially serum-free. In embodiments, the cell culture supplement is substantially albumin-free. In embodiments, the cell culture supplement is substantially serum-free and substantially albumin-free. In embodiments, the supplement is added to a serum-free cell culture medium. In embodiments, the supplement is added to an albumin-free cell culture medium. In embodiments, the supplement is added to a serum-free and albumin-free cell culture medium. In embodiments, the supplement is added to a substantially serum-free and/or substantially albumin-free cell culture medium.

In embodiments, the cell culture medium provided herein includes at least one of a balanced salt solution, basal medium, and/or complex medium. In embodiments, the cell culture medium provided herein includes a balanced salt solution. In embodiments, the cell culture medium provided herein includes a basal medium. In embodiments, the cell culture medium provided herein includes a complex medium.

In embodiments, the cell culture medium provided herein includes at least one of: saline, phosphate-buffered saline, Dulbelcco's phosphate buffered saline, Hank's balanced salt solution, Earle's balanced salt solution, MEM, Opti-MEM, DMEM, CTS KnockOut DMEM, RPMI-1640, IMDM, Ham's F12, F-12 K, F-10, DMEM/F12, Neurobasal, McCoy's 5A medium, Leibowitz's L-15, Medium 199, Neurobasal A, Brainphys, GMEM, and/or William's E Medium. In embodiments, the cell culture medium includes saline. In embodiments, the cell culture medium includes phosphate-buffered saline. In embodiments, the cell culture medium includes Dulbelcco's phosphate buffered saline. In embodiments, the cell culture medium includes Hank's balanced salt solution. In embodiments, the cell culture medium includes Earle's balanced salt solution. In embodiments, the cell culture medium includes MEM. In embodiments, the cell culture medium includes Opti-MEM. In embodiments, the cell culture medium includes DMEM. In embodiments, the cell culture medium includes CTS KnockOut DMEM. In embodiments, the cell culture medium includes RPMI-1640. In embodiments, the cell culture medium includes IMDM. In embodiments, the cell culture medium includes Ham's F12. In embodiments, the cell culture medium includes F-12 K. In embodiments, the cell culture medium includes F-10. In embodiments, the cell culture medium includes DMEM/F12. In embodiments, the cell culture medium includes Neurobasal medium. In embodiments, the cell culture medium includes McCoy's 5A medium. In embodiments, the cell culture medium includes Leibowitz's L-15. In embodiments, the cell culture medium includes Medium 199. In embodiments, the cell culture medium includes Neurobasal A. In embodiments, the cell culture medium includes BRAINPHYS™ medium. In embodiments, the cell culture medium includes GMEM. In embodiments, the cell culture medium includes William's E Medium. In embodiments, the cell culture medium includes a combination of two or more compositions provided herein. In embodiments, one or more of the recited media may be expressly excluded.

In an aspect is provided a cell culture supplement, including: (i) a peptide comprising superoxide dismutase activity and Cu+, Zn+ chelating activity; (ii) vitamin E or an analog thereof; and (iii) a superoxide scavenger. For example, a cell culture supplement as provided herein can include, e.g., Peptide C (SEQ ID NO: 1), Mito-TEMPO, and 6-hydroxy 2, 5, 7, 8, tetramethylchroman carboxylic acid.

In an aspect is provided a serum-free cell culture medium that includes Peptide C (SEQ ID NO: 1), Mito-TEMPO, and 6-hydroxy 2, 5, 7, 8, tetramethylchroman carboxylic acid.

In an aspect is provided an albumin-free cell culture medium that includes Peptide C (SEQ ID NO: 1), Mito-TEMPO, and 6-hydroxy 2, 5, 7, 8, tetramethylchroman carboxylic acid.

### Methods

In an aspect is provided a method for growing cells in culture. The method includes growing the cells in a cell culture medium according provided herein, including embodiments thereof.

In another aspect a method of growing cells in culture is provided. The method includes growing the cells in a cell culture medium, supplemented with a cell culture supplement provided herein including embodiments thereof.

In an aspect a method of rescuing cells from albumin-induced toxicity is provided. The method includes contacting a cell exhibiting albumin-induced toxicity with a cell culture supplement or a cell culture medium provided herein including embodiments thereof.

In an aspect a method for expanding cells in culture is provided. In an embodiment, the method includes contacting the cells with a serum-free, albumin-free cell culture medium in a cell culture medium provided herein including embodiments thereof. In an embodiment, the method includes contacting the cells culture medium supplemented with a cell culture supplement provided herein including embodiments thereof. In an embodiment, the cell culture medium is serum free and/or albumin free.

In an aspect is provided a method for recovering cells from oxidative stress. The method may include contacting the cells with a cell culture supplement provided herein including embodiments thereof. The method may include growing the cells in a cell culture medium provided herein including embodiments thereof. In embodiments, the oxidative stress is from freezing the cells. In embodiments, the oxidative stress is exposure to a lipid rich condition.

For the methods provided herein, in embodiments, the cells are therapeutic cells. In embodiments, the cells are cells used for production of a biologic. In embodiments, the cells are cells used for production of a therapeutic peptide. In embodiments, the cells are eukaryotic cells. In embodiments, the cells are used as a cell therapy. In embodiments, the cells are mammalian cells. In embodiments, the cells are rodent cells. In embodiments, the cells are primate cells. In embodiments, the cells are human cells. In embodiments, the cells are stem cells, such as embryonic stem cells, induced pluripotent stem cells, or the like. In embodiments, the cells are embryonic stem cells. In embodiments, the cells are induced pluripotent stem cells. In embodiments, the cells are immune cells, such as B cells, T cells, NK cells, or the like. In embodiments, the cells are B cells. In embodiments, the cells are T cells. In embodiments, the cells are NK cells.

In embodiments, the cells are stem cells. In embodiments, the cells are embryonic stem cells. In embodiments, the cells are pluripotent stem cells. In embodiments, the cells are iPSCs. In embodiments, the cells are progenitor cells. In embodiments, the cells are immortalized cells. In embodiments, the cells are primary cells. In embodiments, the cells are a cell line. In embodiments, the cells are manufacturing cells. In embodiments, the cells are selected from: mesenchymal stem cells, iPSCs, hESCs, neural progenitor cells, retinal pigment epithelium, pancreatic beta cells, cardiac muscle cells, HEK-293 cells, and CHO cells. In embodiments, the cells are mesenchymal stem cells. In embodiments, the cells are neural progenitor cells. In embodiments, the cells are retinal pigment epithelial cells. In embodiments, the cells are pancreatic beta cells. In embodiments, the cells are cardiac muscle cells. In embodiments, the cells are HEK-293 cells. In embodiments, the cells are CHO cells. In embodiments, one or more of the recited cell types may be expressly excluded.

### Kits

In an aspect is provided a cell culture kit including a serum-free cell culture medium and a cell culture supplement provided herein including embodiments thereof. In embodiments, the cell culture supplement includes: (i) a peptide having superoxide dismutase activity and Cu+, Zn+ chelating activity; (ii) vitamin E or an analog thereof; and (iii) a superoxide scavenger, and wherein (i)-(iii) are provided in two or more separate containers. In embodiments, the cell culture supplement includes: (i) a peptide comprising superoxide dismutase activity and Cu+, Zn+ chelating activity; (ii) vitamin E or an analog thereof; and (iii) a superoxide scavenger, and wherein (i)-(iii) are provided in a single container.

In embodiments, the kit further includes a hydrogen peroxide reducing reagent. In embodiments, the hydrogen peroxide reducing reagent includes glutathione and/or lipoic acid, or variant thereof. In embodiments, the hydrogen peroxide reducing reagent includes glutathione. In embodiments, the hydrogen peroxide reducing reagent includes lipoic acid. In embodiments, the hydrogen peroxide reducing reagent includes a variant of glutathione. In embodiments, the hydrogen peroxide reducing reagent includes a variant of lipoic acid.

In an aspect is provided a cell culture kit including a cell culture medium including a peptide having superoxide dismutase activity and Cu+, Zn+ chelating activity, as provided herein including embodiments thereof, and one or more cell culture supplements. In embodiments, the one or more cell culture supplements include vitamin E or an analog thereof, a superoxide scavenger, or a hydrogen peroxide reducing reagent. In an embodiment, the one or more cell culture supplements are provided in a single container. In an embodiment, the one or more cell culture supplements are provided in two or more containers.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

One skilled in the art would understand that descriptions of making and using the particles described herein is for the sole purpose of illustration, and that the present disclosure is not limited by this illustration.

### Example 1. Differential Components of Albumins Affect Cell Culture Performance

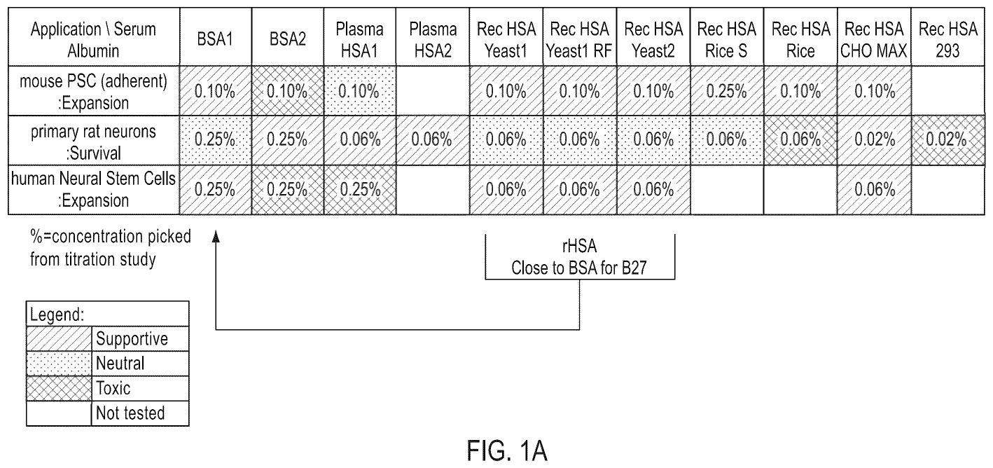

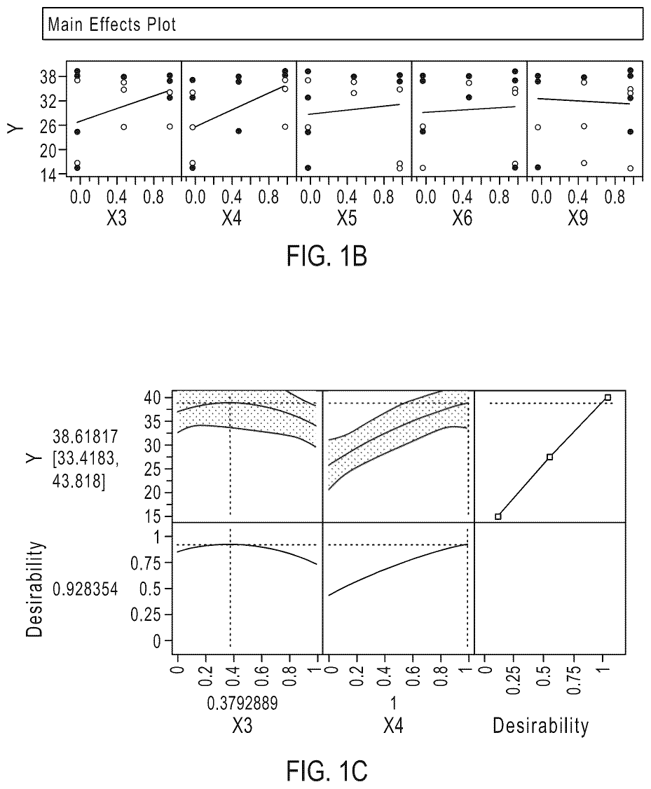

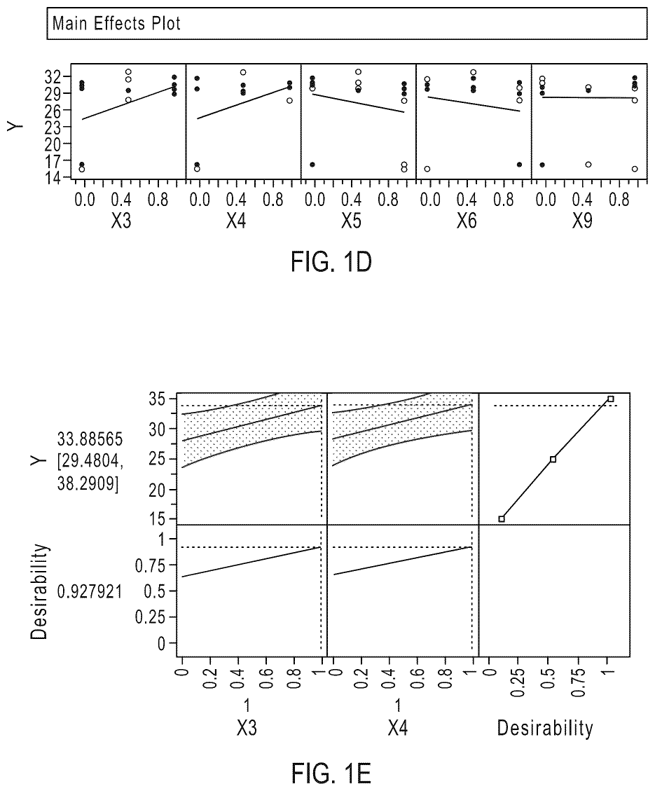

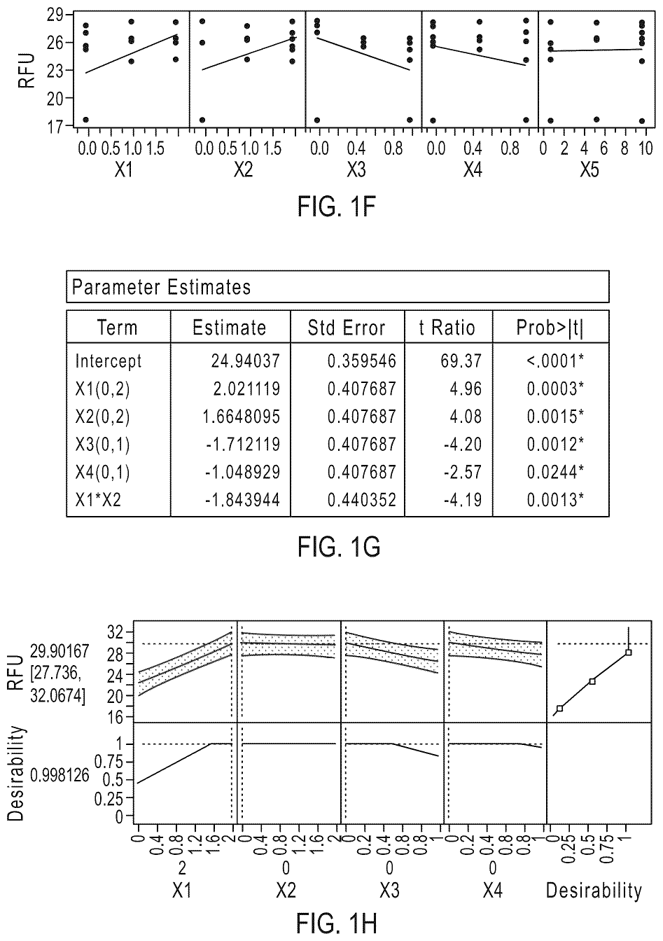

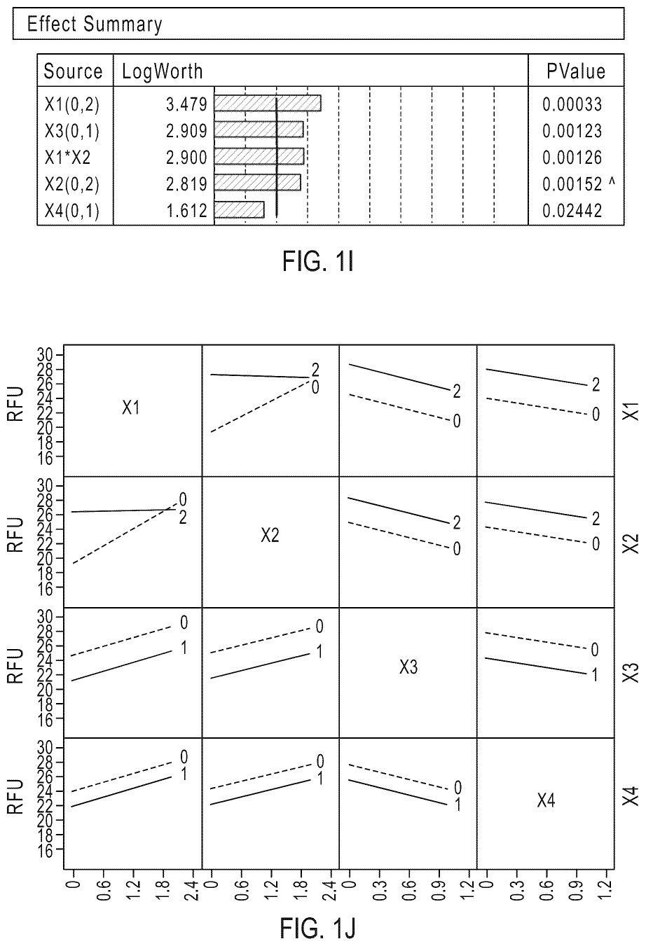

Commercially available albumins were analyzed for performance in cell cultures. Bovine serum albumin (BSA), human serum albumin (HSA) and recombinant human serum albumin (Rec HSA) from various sources (including different manufacturers, bovine, human, recombinant HSA (made in yeast or rice), and Freestyle CHO MAX media (Thermo Fisher cat. no. K900020) were analyzed. B-27 (Thermo Fisher Scientific cat. no. 17504044) supplemented cell cultures for mouse pluripotent stem cells (PSC), primary rat neurons, or human neural stem cells (NSC) were prepared (approx. 0.5 to 1× final concentration of B27 in medium). The cultures varied only in the source or type of albumin present. Results shown in FIG. 1A illustrate that significant performance differences existed among the albumins tested, and further that performance is dependent on cell type. In fact, two cultures comprising the same cell line and differing only in addition of BSA1 or BSA2 differed in performance. BSA1 showed supportive properties in mouse PSC and human NSC cultures, while addition of BSA2 caused toxicity in both cell cultures. Further, the recombinant (rec) HSA samples tested differed in that certain samples added to cultures had a neutral effect on primary rat neuron cells, one was supportive, and two caused toxicity in the cells. These results illustrate that the type and source of albumin both have significantly differential effects on cell culture performance, and that these effects differ by cell type.

Next, interactions between different components of B27 supplement and various albumins were analyzed (FIGS. 1B-1J). The effect of these molecular interactions were also assessed for their effect on neuronal cell stability in cell culture. For example, both BSA and HSA interacted with B27 components X1 (Tocopherol) and X2 (Tocopherol acetate), which showed positive effects on neuronal cell viability. Further, X1 and X2 interactions with BSA became saturated when higher concentrations of BSA were titrated into culture. Conversely, the same concentrations of rHSA added to the culture did not result in saturation with X1 and X2 components. Moreover, interactions of BSA with X3 (Linoleic acid) and X4 (Linolenic acid) components positively affected neuronal viability in B27-supplemented media, while addition of rHSA negatively affected viability in culture. Results are illustrated in FIGS. 1B-1E. When a broad range of rHSA was tested in B27 supplemented cultures, it was shown that component X1 and competed with component X2 for rHSA binding. On the contrary, component X5 did not interact with rHSA, even at high concentrations, as illustrated in FIGS. 1F-1J. These results collectively indicate that various albumins have different interactions with cell culture media components, thereby exerting variance on culture performance.

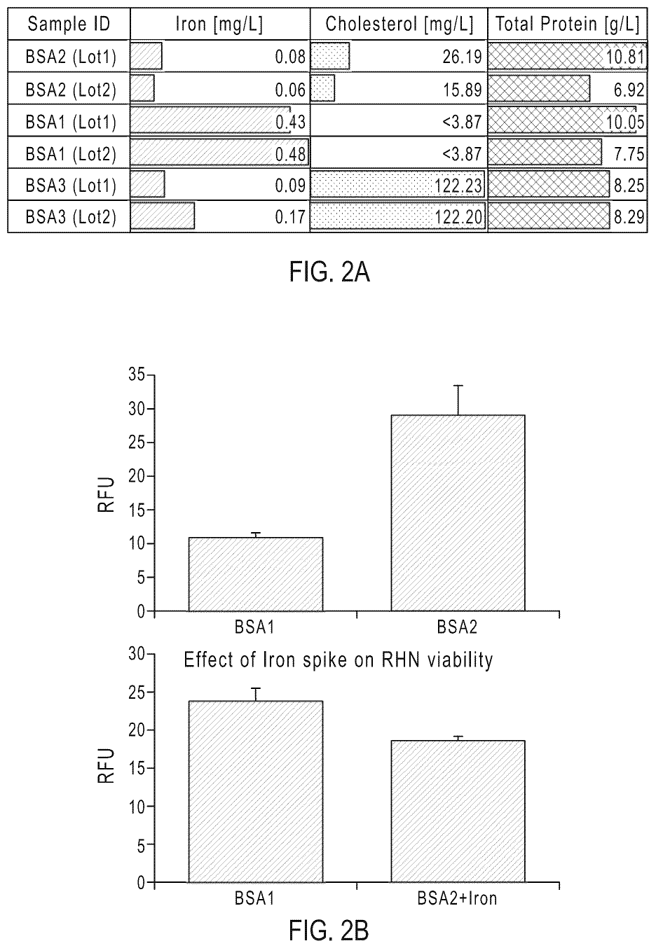

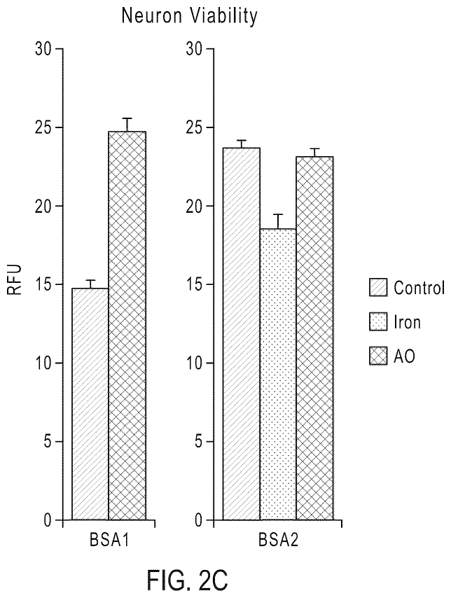

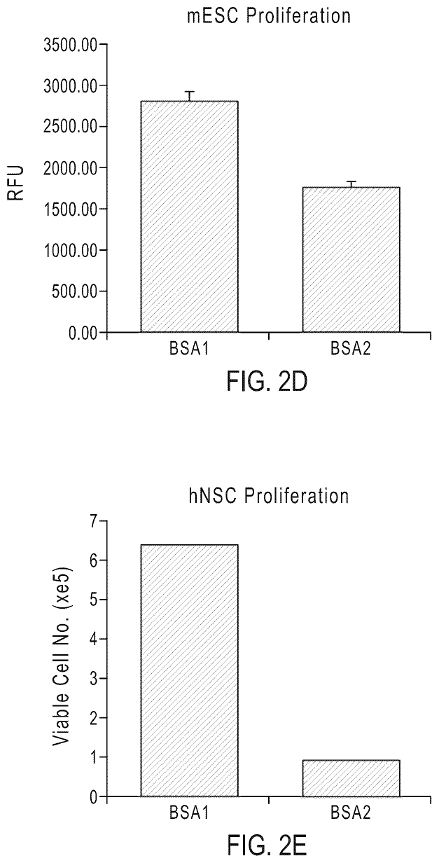

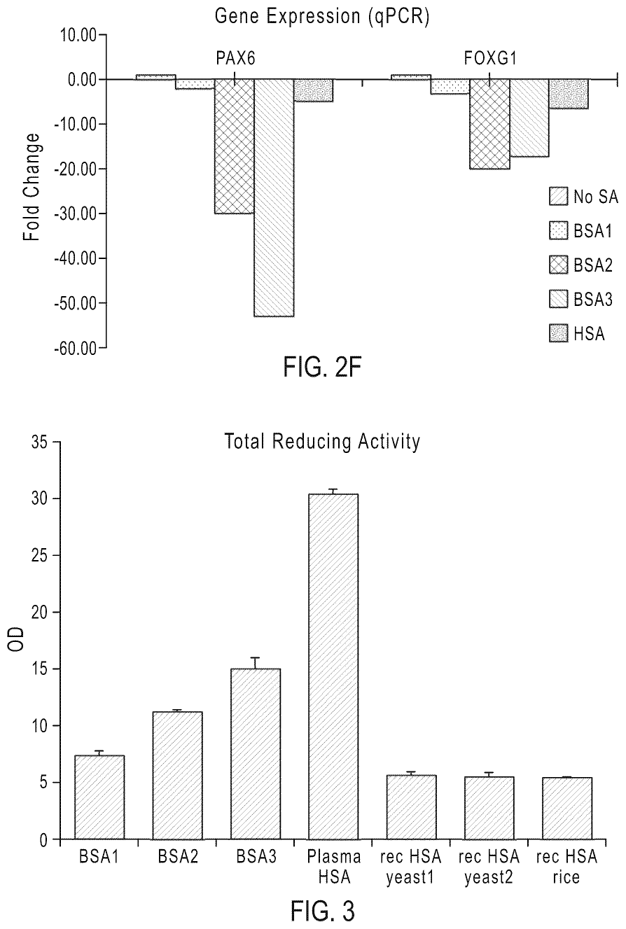

The effect of BSA from three sources, BSA1, BSA2, and BSA3 were analyzed for their performance in rat neuron survival. BSA1 included higher iron content compared to BSA2 and BSA3, while BSA3 had higher cholesterol content than either of the other two (FIG. 2A). Lot-to-lot variability in iron and cholesterol content (i.e., different lots from the same source) was low. Total protein varied between sources and lots. These data demonstrate that the types and amounts of contaminants in albumin varies between sources, and even between lots.

The effect of different BSAs on cell viability was tested using the eBioscience™ Calcein AM Viability Dye, according to manufacturer's protocol. Briefly, rat neurons were cultured in neurobasal medium supplemented with ITS and BSA1 or BSA2 at a concentration of 0.25%. As shown in FIG. 2B (top panel), results from a Calcein AM viability assay (Thermo Fisher Scientific, Waltham, MA, Cat. No. 65-0853-78) illustrated that BSA1 (which has a higher iron content compared to BSA2), reduced viability of rat neurons compared to rat neurons grown in the presence of BSA2. When the culture including BSA2 was spiked with iron, rat neuron viability was decreased, as illustrated in the results of FIG. 2B (bottom panel). These results indicate that albumins that include higher iron content have a toxic effect on neuron cell viability.

The ability of antioxidant to reverse iron-induced toxicity was then investigated. Tocopherol at a concentration of 2 μg/mL in neurobasal medium supplemented with lean supplement (Insulin-Transferrin-Selenium, ITS) was added to cultured rat neuron cells and either BSA1 or BSA2. Addition of antioxidant reversed decreased viability of the rat neuron cells due to BSA1 (FIG. 2C, left) or iron (FIG. 2C, right). These results indicate that neuronal stress due to iron can be reversed by addition of antioxidant.

BSA1 and BSA2 were further analyzed for their effect on stem cells. Mouse embryonic stem cells (mESC) or human neural stem cells (hNSC) were grown in cell culture media containing BSA1 or BSA2, and proliferation was measured. Proliferation of mESCs was measured using PrestoBlue assay (Thermo Fisher Scientific) according to the manufacturer's protocol. Proliferation of hNSCs was measured with VI-CELL™ XR cell counting assay (Beckman Coulter) according to the manufacturer's protocol.

As shown in FIGS. 2D and 2E, BSA2 reduces proliferation of both mESC and hNSC when compared to proliferation of the same cells cultured in the presence of BSA1. These results show that differences between BSA samples will result in variations in their supportive role in cell cultures, thus significantly impacting culture performance.

Cholesterol concentrations differ among BSA from disparate sources (see FIG. 2A). Without wishing to be bound by theory, variance in gene expression involved in cell differentiation may occur in cells cultured in media comprising cholesterol, since cholesterol is an agonist of the Shh pathway. Cell were differentiated in Essential 6 media and Forkhead box protein G1 (FOXG1) and Paired box protein (PAX-6) expression was examined to analyze the effect of albumin on gene expression. Expression levels of FOXG1 and PAX-6, which are downstream of Shh were measured by qPCR in hPSCs cells cultured with either BSA1 (fatty acid free), BSA2 (fatty acid rich), or BSA3 (fatty acid rich). Results are shown in FIG. 2F. The results show that BSA from different sources have different effects on Pax6 and FoxG1 gene expression, possibly due to variations in contaminant cholesterol among the albumin samples tested. Thus, levels of lipid contaminants in albumins can impact pluripotent stem cell differentiation.

These results collectively show that undefined contaminants accompany albumins, and differentially affect cell culture performance. Therefore, elimination of albumins from cell cultures may reduce the risk of contaminants, particularly contaminants of blood origin, and other undefined factors.

### Example 2: Antioxidant properties of albumins and chemically-defined antioxidant substitutes thereof

The experiments described herein were performed to explore the physiological functions of albumins and to explore replacement of albumins with chemically defined components. First, antioxidant properties of albumins was analyzed. Total antioxidant power of various albumins was measured by the using the Ferric Antioxidant Status Detection Kit (Thermo Fisher Scientific, Waltham, MA), according to the manufacturer's protocol. The results shown in FIG. 3 indicate that anti-oxidant activity differs significantly among the albumins tested. The results further show that human plasma serum albumin has significantly higher level of reducing power compared to recombinant HSAs. As the use of plasma in culture media is source of blood origin contaminants, the elimination of serum and plasma is highly desirable.

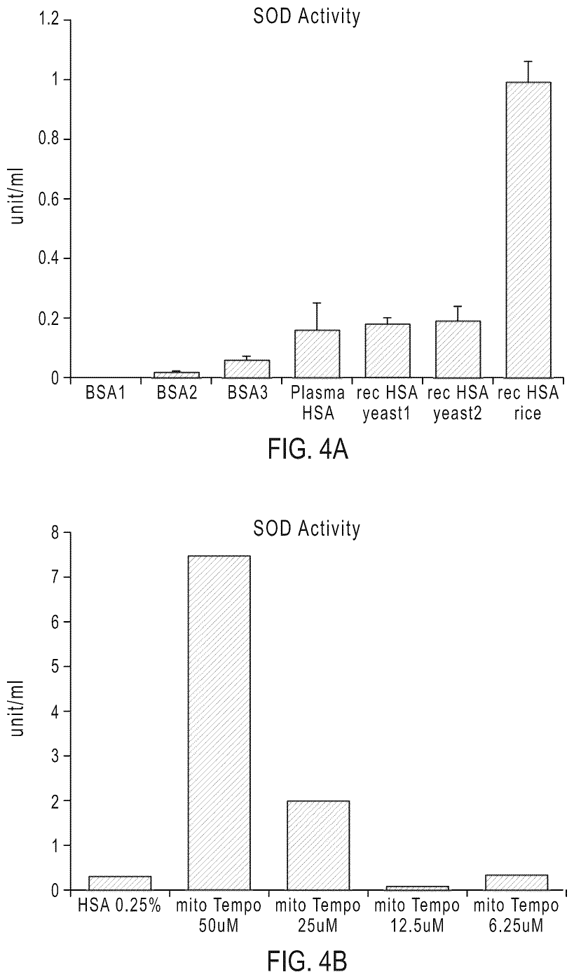

Various classes of antioxidant activity in albumins were investigated. Applicants first analyzed the superoxide dismutase (SOD) activity of various albumins, utilizing the Superoxide Dismutase (SOD) Colorimetric Activity Kit (Thermo Fisher Scientific, Waltham, MA), according to the manufacturer's protocol. Briefly, SOD activity was assessed utilizing an assay comprising the reaction of a substrate and Xanthine Oxidase to form superoxide (O⁻₂), the levels of which are decreased by SOD activity. As shown in FIG. 4A, albumins were confirmed to have SOD activity, and moreover, the SOD activities in the analyzed albumins were significantly different. Notably, recombinant HSA derived from rice displayed highest antioxidant activity.

Next, the ability of chemically defined components to replace SOD antioxidant activity was tested. Mito-Tempo, which scavenges O2•⁻ in mitochondria, was compared with HSA for its ability to reduce superoxide, as assessed using the Superoxide Dismutase (SOD) Colorimetric Activity Kit (Thermo Fisher Scientific, Waltham, MA), according to the manufacturer's protocol. As shown in FIG. 4B, Mito-Tempo concentrations decrease superoxide levels in a dose-dependent manner. Thus, this compound may be used to replicate and replace albumin SOD-like antioxidant functionality.

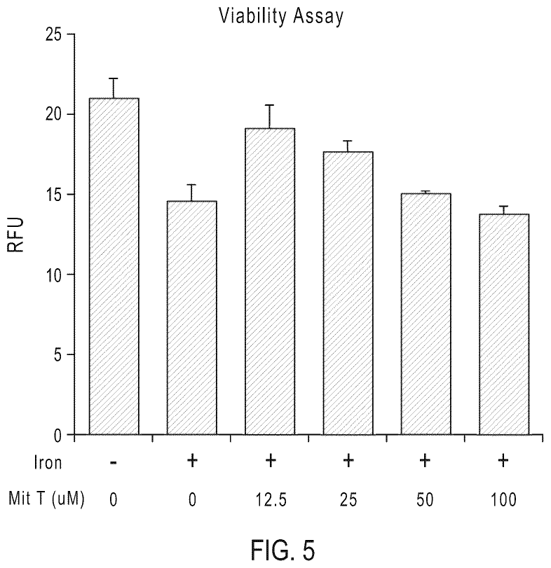

The ability of Mito-Tempo to rescue cultured cells from oxidative stress was tested. Primary rat neuron cells are particularly vulnerable to ROS, and thus served as a model for assessing whether Mito-Tempo provided a beneficial effect on cell cultures. Primary rat neuron cells were cultured in neurobasal medium supplemented with ITS in the presence of 0.12 μg/mL iron. At day 6, Mito-Tempo was titrated into the culture medium at the indicated concentrations. After 6 days in culture, cell viability was measured with Calcein AM staining. As shown in FIG. 5, Mito-Tempo was effective in rescuing cells up to a concentration of about 12.5 to about 25 μM. These results indicate that Mito-Tempo can replace the functionality of albumins for rescuing cells from ROS related stress.

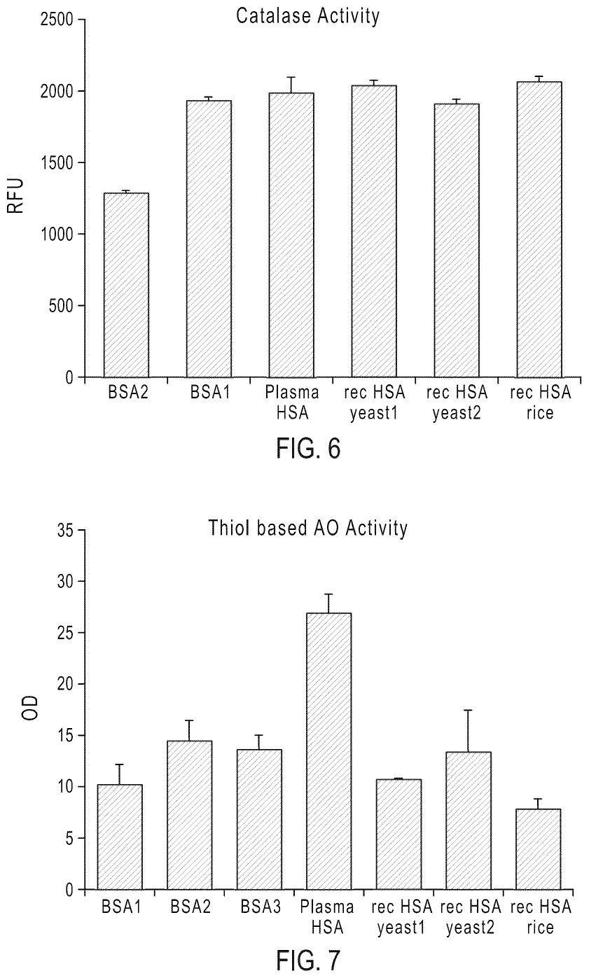

The catalase activity of albumins was then identified and measured by Amplex™ Red Hydrogen Peroxide/Peroxidase Assay (Thermo Fisher Scientific) according to the manufacturer's protocol. Briefly, hydrogen peroxide and catalase forms products that when mixed with Amplex Red and horseradish peroxidase forms the fluorescent product Resorufin. Thus, intensity of fluorescence signal can be used to measure catalase activity levels. Different sources of BSA, recombinant HSA, or plasma HSA, as indicated were assessed utilizing this method. Each of the albumins tested had catalase activity, although levels differed among albumin samples (FIG. 6). Particularly, the two BSA samples tested showed significant variation in reductive activity.

Albumin has a cysteine residue at position 34, which exists either as a free thiol or may form a disulfide bond with glutathione (GSH). Because glutathione peroxidase uses GSH as a reductant to remove H₂O₂, a powerful oxidizing agent that is potentially damaging to cells, albumins play a pivotal role in regulating intracellular levels of GSH. Using Ellman's reagent (DTNB, 5,5-dithio-bis-(2-nitrobenzoic acid)), which produces 1,3,5-Trinitrobenzene (TNB) in the presence of thiols, various albumins were tested for thiol-based antioxidant activity. Results show that GSH based antioxidant activity was highest in plasma HSA (FIG. 7). Further, activity levels differed among the types and sources of albumins analyzed.

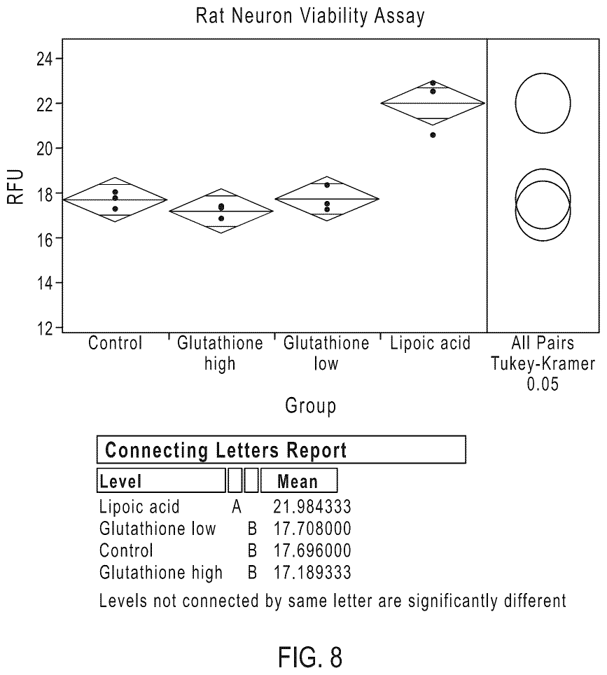

Applicants then tested chemically defined components that may replace the catalase and thiol antioxidant activity of albumins. GSH and lipoic acid were assessed for their ability to provide catalase and thiol antioxidant activity by culturing rat neurons in the presence of either low or high concentrations of GSH or lipoic acid and subsequently assaying cell viability. Lipoic acid enhanced rat neuron cell viability, compared to a control culture (FIG. 8), and therefore may be used to replace the catalase and thiol activity of HSA. Conversely, neither concentration of GSH tested showed significant effects on cell viability compared to the control.

### Example 3. Albumin Metal Chelating Properties and Chemically Defined Peptide Replacements Thereof

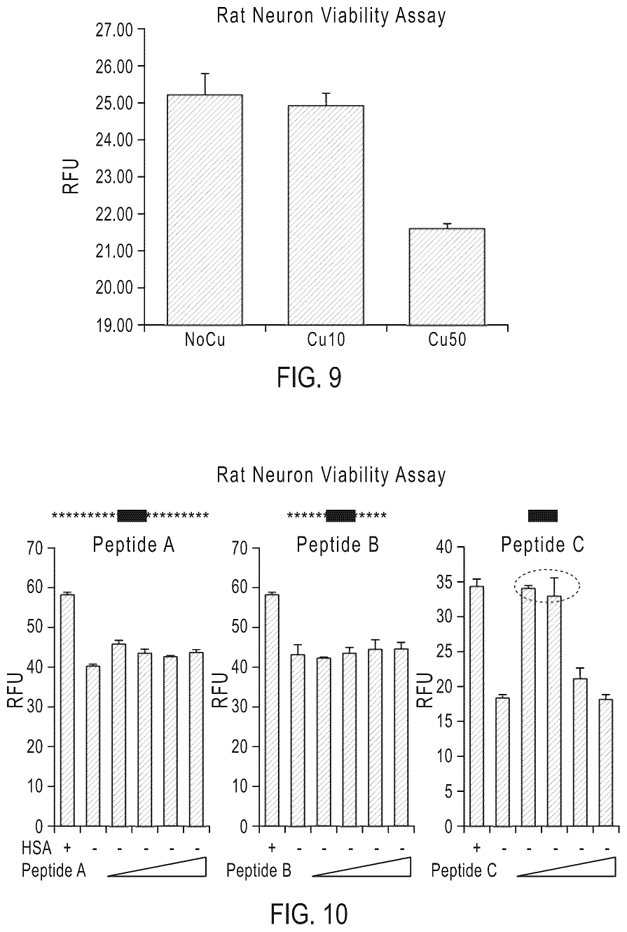

Free redox active transition metal ions, including iron and copper, can be pro-oxidant and participate in reactions, including Fenton and Haber Weiss reactions, to produce hydroxyl radicals OH^{•}. The presence of radicals in cell cultures damages cells and decreases culture performance. For example, rat neuron cells cultured in the presence of copper (10 μM or 50 μM, Cu10 and Cu50, respectively), show reduced viability compared to cells cultured in the absence of the metal (NoCu), as illustrated in FIG. 9. Albumins may bind to metals to control reactivity and limit availability, thereby reducing damage caused by OH^{•} radicals.

Albumins such as HSA have a metal binding site, which functions as a chelator of transition metals that are toxic to neuron cells. Applicants therefore investigated whether HSA may be replaced by a synthetic peptide, which mimics the high affinity binding of the functional metal binding site. Results illustrated in FIG. 10, show that certain concentrations of the HSA metal binding site comprising the chelating amino acid sequence DAHK (Peptide C; SEQ ID NO:1) enhanced rat neuron cell viability when grown in culture medium lacking albumin, mimicking the supportive features of HSA. Surprisingly, peptide A, which is the Peptide C sequence extended to include 10 additional HSA residues on both the N-terminal and C-terminal ends did not retain HSA supportive properties. Peptide B, which is the Peptide C sequence extended by 5 additional flanking HSA residues on either end, was similarly non-functional. These results indicate that Peptide C retains metal chelating ability on its own, though its function is impacted by neighboring amino acid residues. Without wishing to be bound by scientific theory, the additional amino acid residues may block or otherwise inhibit the chelating functions of the Peptide C sequence. Further, these data indicate that the peptides described herein (including Peptide C), can replace albumin in culture medium (e.g, when provided as a supplement or as part of a complete culture medium).

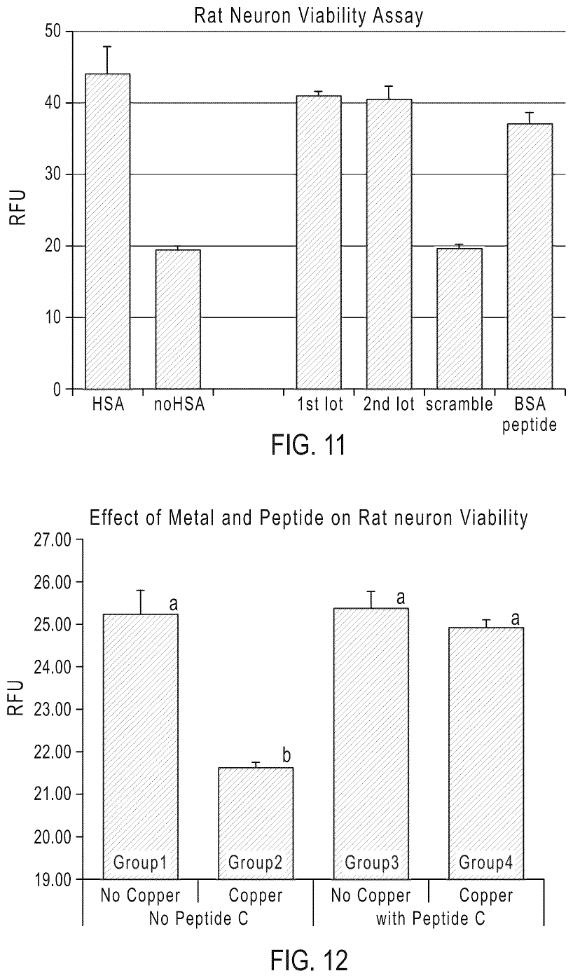

The importance of Peptide C ionic property and sequence specificity in conferring enhancing properties to cell cultures was investigated. The tetrapeptide DAHK (SEQ ID NO:1) is a sequence comprising negatively, neutral, and two positively charged amino acids (−X++). The counterpart BSA peptide comprising the sequence DTHK (SEQ ID NO:2) notably has the same order of charged amino acids as Peptide C. Similar to Peptide C (1^{st} lot and 2^{nd} lot), DTHK (SEQ ID NO: 2; BSA peptide), was shown to be functional in improving viability of cultured rat neuron cells, as illustrated in FIG. 11. To assess if amino acid sequence or charge specificity confers beneficial properties, a scrambled Peptide C sequence was prepared and tested. The scrambled peptide comprising the sequence AKDH (SEQ ID NO: 3; scramble) and charge of X+−+ was tested, and results are shown in FIG. 11. The results suggest that the culture supportive function of the tetrapeptides is related to the specific combination of charged amino acids. For example, the charge sequence of −X++ conferred cell culture enhancing properties, but when the charge sequence was scrambled, the HSA-like function was abolished.

Applicants further tested multiple lots of the HSA peptide DAHK (SEQ ID NO: 1), and results shown in FIG. 11 illustrate that the peptide demonstrated lot-to-lot consistency and good shelf life, even in solution. These results collectively indicate that synthetic peptides may replace human serum albumins in cell cultures as a supportive supplement.

Copper levels must be regulated for optimal cell viability, and cell cultures including copper consistently resulted in toxicity for rat neuron cells. Therefore, Peptide C was investigated to confirm its ability to retain HSA metal chelating activity. Primary rat neuron cells were cultured in neurobasal medium supplemented with ITS, in the presence of 50 μM copper resulted in toxicity for the cells. However, addition of Peptide C in the culture prevented toxicity even in the presence of copper, as shown in FIG. 12. In fact, rat neuron cells cultured in the presence of copper and Peptide C showed similar levels of live, healthy cells compared to cultures without copper. The results illustrate that Peptide C retains HSA functionality as a transition metal chelator. Therefore, synthetic peptides may replicate and replace metal binding activity of albumins in cell cultures and other biological assays.

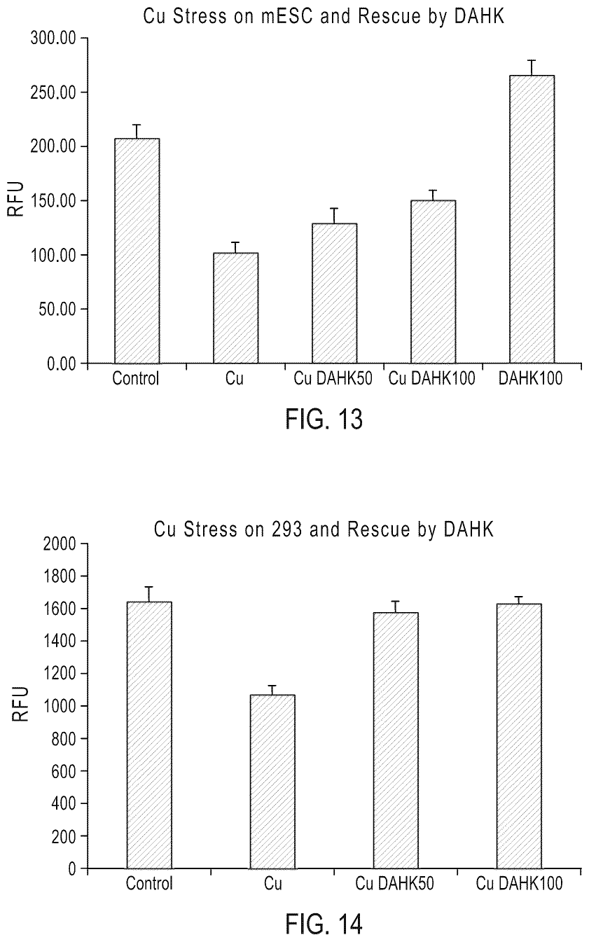

The effect of metals and synthetic peptides were further assessed in mouse embryonic stem cell (mESC) and HEK293 cell proliferation cultures. mESC and HEK293 cultures were spiked with 50 μM copper, thereby creating a toxic culture environment and inhibiting cell proliferation. Addition of either 100 μg/mL or 50 μg/mL of Peptide C into the cultures rescued the stressed cells in a concentration dependent manner, particularly for mESC cells, and to a lesser extent HEK293 cells. PRESTOBLUE™ cell viability reagent (Thermo Fisher Scientific, Waltham, MA), was used to assess viability according to the manufacturer's protocol. The viability assays showed that Peptide C both rescued copper induced stress and enhanced cell proliferation, as shown in FIGS. 13 and 14. The results indicate that Peptide C may be a supplement to serum-free and lean cultures (e.g. Essential 8) to improve media stability and quality, and can replace HSA for metal chelating ability.

Because Peptide C displays beneficial properties in cell culture as described herein, Applicants further tested various Peptide C derived synthetic peptides for their ability to replace albumins in cell culture. To determine the impact of peptide length, Applicants assessed a variety of peptides that varied both in length and in the identity of the amino acid residue(s) that flank the Peptide C DAHK sequence (SEQ ID NO:1). Further, to assess the effect of ionic property, peptides were tested that included amino acid substitutions to the peptide of SEQ ID NO:1. The amino acid substitutions were designed to retain ionic and hydrophobic properties of SEQ ID NO:1, namely by preserving the order of negatively charged, hydrophobic uncharged, and positively charged residues. Thus, primary rat neurons were cultured in neurobasal medium supplemented with ITS. The cultures were further supplemented with rHSA, or a variation of the Peptide C derived synthetic peptide.

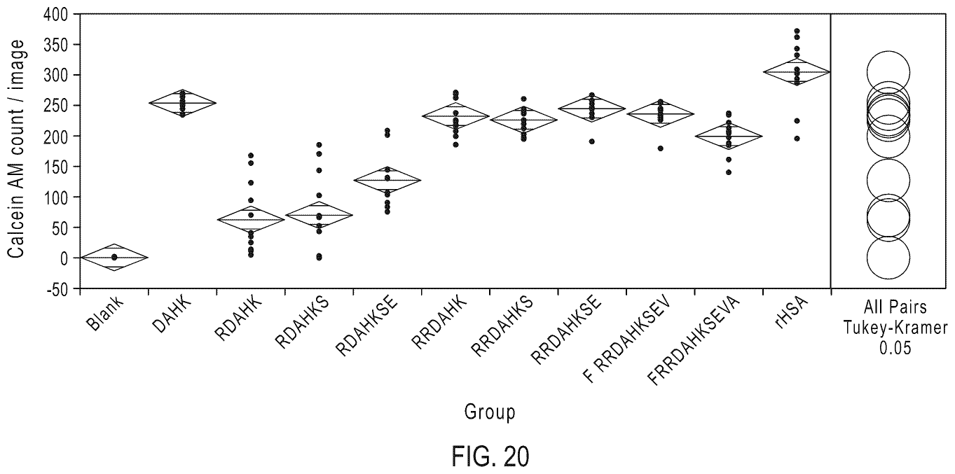

In a first set of cell cultures, the synthetic peptides included the DAHK (SEQ ID NO:1) amino acid sequence or peptides with additional residues on one or both the N-terminus and C-terminus of SEQ ID NO:1. On day 5 of culturing, the cells were labeled with Calcein AM and counted. Results illustrated in FIG. 20 show that peptides having the sequence of SEQ ID NO:12, SEQ ID NO:13, SEQ ID NO:15, SEQ ID NO:16 and SEQ ID NO:17 convey beneficial properties to cell culture and thus may replace or partially replace albumins, for example HSA in cultures. The results indicate that synthetic peptides with additional residues on one or both sides of SEQ ID NO:1 may be used in cultures to at least partially replace albumin if an active conformation of peptide C is maintained. That is, the length of adjacent residues on the N-terminus and C-terminus of the peptide is not critical for maintaining the function of peptide C unless the conformation is impacted as so not to maintain its beneficial properties in cell culture.

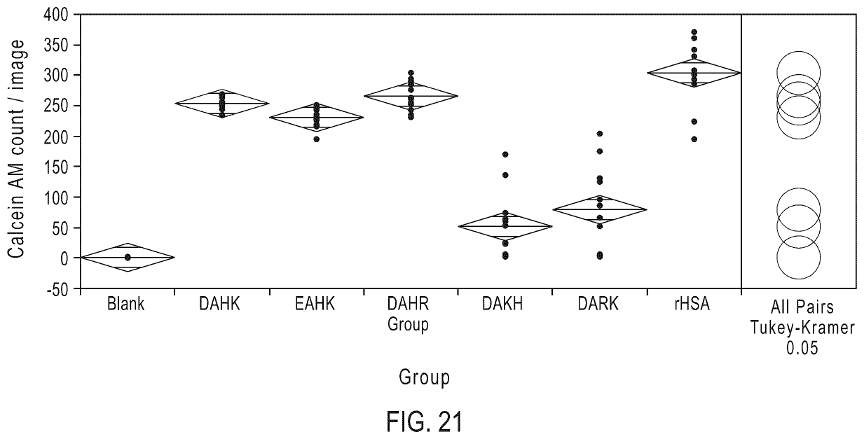

In a second set of cultures, the rat neuron cells were cultured with synthetic peptides that include the peptide DAHK (SEQ ID NO:1) and derivatives which preserve the ionic and hydrophobic properties of the DAHK peptide (SEQ ID NO: 1). Results shown in FIG. 21 show that on in addition to ionic properties, the identity of the third amino acid is particularly important. For example, the first amino acid may be occupied with either of negatively charged amino acid of D or E and the second amino acid can be a non-charged amino acid, such as A or T. Further, the fourth amino acid can be a positively charged amino acid, such as K or R. However, the third amino acid must be an H residue and cannot be replaced by other positively charged amino acids, for example K or R.

### Example 4. Synthetic Peptide Replacements for Albumins in Stem Cell Cultures

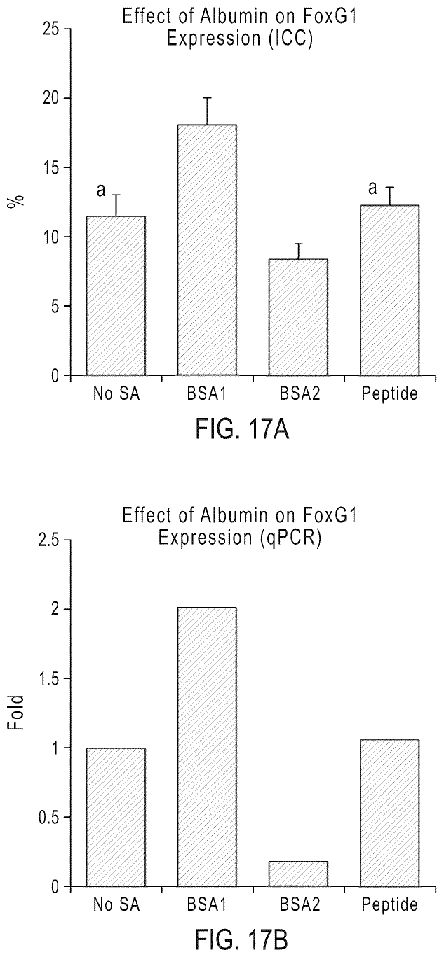

Peptide C (SEQ ID NO:1) was assessed for its effect on cell differentiation. As described herein, the presence of BSA from various sources resulted in inconsistent expression levels of genes involved in cell differentiation in cultured cells. Without wishing to be bound by theory, this was likely due to variations in cholesterol levels within the albumin samples. To assess the effect of the tetrapeptide Protein C on cell differentiation, pluripotent stem cells (PSC) were cultured in the presence either BSA1, BSA2, or peptide C. PSC cultured with Peptide C resulted in similar expression levels of FoxG1 as compared with a control culture (No SA), as determined by immunocytochemistry (ICC) and qPCR. Results are shown in FIGS. 17A and 17B, respectively. Further analysis of Peptide C confirmed that the peptide does not include any cholesterol contaminants. Upon differentiation, cells will develop their positional identity such as forebrain, midbrain, hindbrain etc. Specification is the step where PSC develop this positional identification. The data herein show that specification of PSC differentiation was impacted the presence of BSA1 or BSA2 in the cultures, whereas there no significant impact was shown with Peptide C. These results indicate that synthetic peptides may replace albumins in stem cell cultures to prevent variations in gene expression resulting in inconsistent cell differentiation.

### Example 5. Vitamin E Antioxidant Activity in Albumins and Chemically Defined Substitutes Thereof

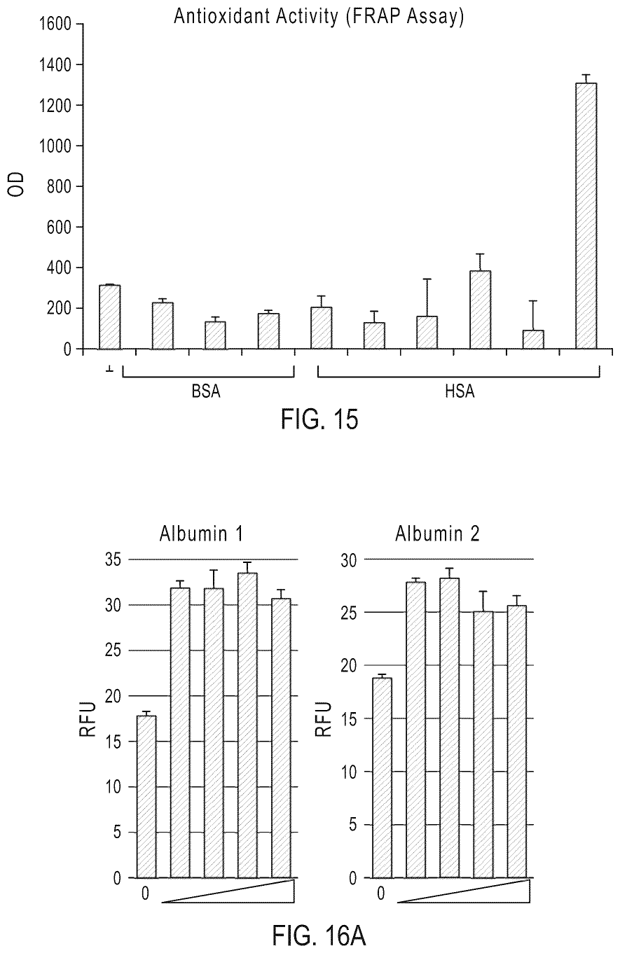

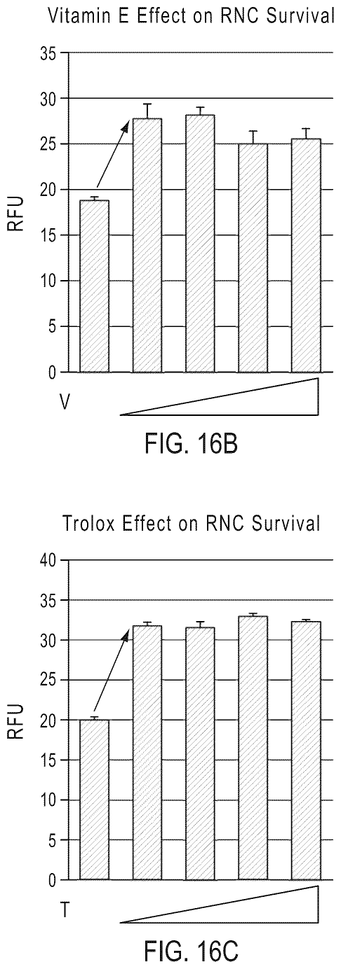

As described herein, albumins confer antioxidant activity, though the activity is variable and inconsistent between albumin homologs, sources, and lots. Further, as confirmed by HPLC and shown in FIG. 15, antioxidant components vary in their concentration and activity between albumin samples. Because fat soluble vitamins, which have antioxidant properties, are known to bind albumin, two types of albumin were assessed for their Vitamin E content. A low fat albumin (albumin 1) and high fat albumin (albumin 2) were analyzed by titrating Vitamin E into the samples. Results shown in FIG. 16A indicate that Vitamin E saturation occurred at lower concentrations for albumin 2 compared to albumin 1. These data suggest that albumin 2 had a higher level of Vitamin E (or agonist of vitamin E) compared to albumin 1.

Vitamin E belongs to a class of lipophilic antioxidants which are efficient scavengers of ROS and lipid radicals, making them indispensable protectors and essential components of biological membranes. Thus, Applicants sought to identify and characterize a chemically defined component which may replicate the properties of Vitamin E in albumin.

Trolox (6-hydroxy-2,5,7,8-tetramethylchroman-2-carboxylic acid), a water-soluble derivative of Vitamin E, was investigated as a replacement for albumin. Rat cortical neuron (RCN) cells where cultured in neurobasal medium supplemented with ITS, in the presence of increasing concentrations of Vitamin E or Trolox. Without any anti-oxidant, aged neurons started to degenerate at about day 4, leaving few viable neurons left on the plate. Surprisingly, titration of either Vitamin E (FIG. 16B) or Trolox (FIG. 16C) into the cell culture reversed cell death as assessed on day 6. Further, Trolox has higher water solubility and wider working range than Vitamin E, making it a preferred culture supplement over the vitamin. These results indicate that Trolox is a viable substitute for the Vitamin E activity of albumins for preventing cell degeneration.

### Example 6. Compositions Including Chemically Defined Albumin Substitutes

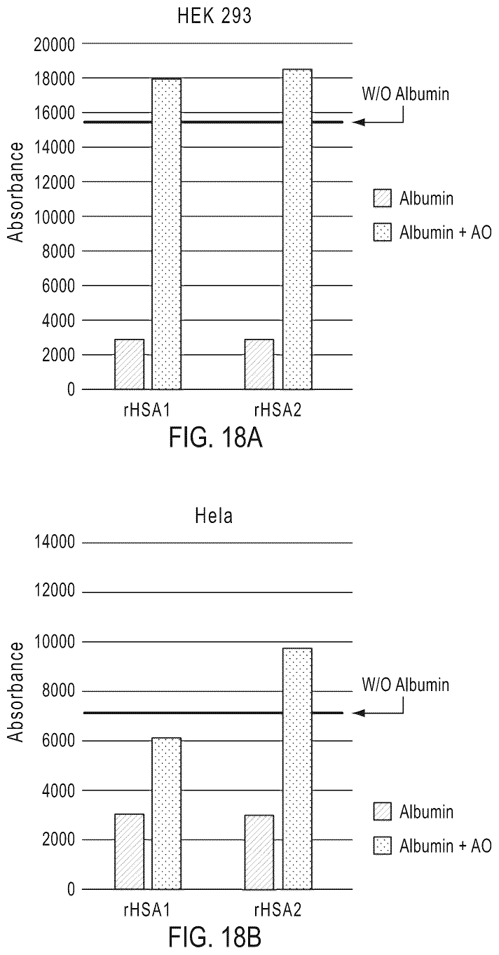

The antioxidants provided herein were assessed in compositions including albumin. Applicants investigated whether antioxidants may confer beneficial properties to cell cultures, in addition to reversing toxic features of albumins when used in combination. Recombinant human serum albumin derived from rice (rHSA), which has exhibited toxicity towards cells in culture, was tested in cell cultures either in the presence or absence of antioxidants. HEK-293 cells, when assessed with a PRESTOBLUE™ cell viability reagent (Thermo Fisher Scientific, Waltham, MA) according to the manufacturer's protocol. The data show higher levels of proliferation when cells are cultured with rHSA compared to cells cultured in the absence of additional antioxidants, as illustrated in FIG. 18A. Hela cells showed similar results, as shown in FIG. 18B. Surprisingly, antioxidant improved cell proliferation compared to control groups of cells cultured in the absence of rHSA. These results indicate that chemically defined anti-oxidants may be combined with albumins to both confer beneficial properties and to rescue toxicity from albumin contaminants.

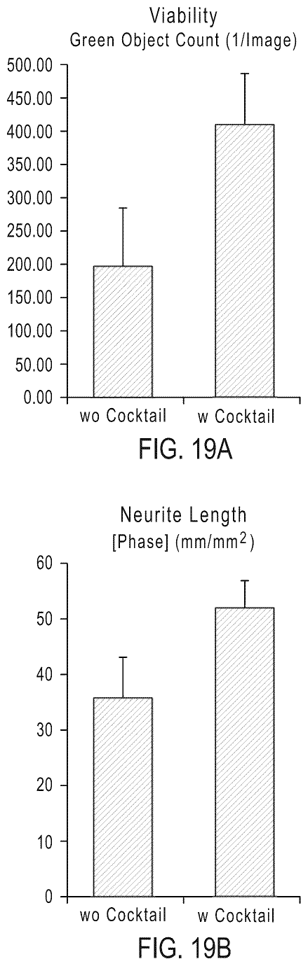

Further, a cocktail comprising Peptide C and antioxidant was analyzed for its ability to support cell growth in culture. Neuron cells were cultured in N2 media supplemented with Transferrin (holo). The cells were cultured either in the presence or absence of the cocktail. Live cells were subsequently labeled with Calcein AM and counted. Results illustrated in FIG. 19A indicate that in the presence of Peptide C and antioxidant, neuronal cells showed greater viability than cells cultured in the absence of cocktail. Further, images of Calcein AM labeled cells and analysis of neurite length show that the cocktail enhanced neurite growth and formation, as illustrated in FIG. 19B. These results indicate that compositions including Peptide C and other chemically defined albumin substitutes may be used to improve cell culture performance.

Informal Sequence Listing

| | |
|---|---|
| SEQ ID NO: 1 | DAHK |
| SEQ ID NO: 2 | DTHK |
| SEQ ID NO: 3 | AKDH |
| SEQ ID NO: 4 | GVFRRDAHKSEVAH |
| SEQ ID NO: 5 | SAYSRGVFRRDAHKSEVAHRFKD |
| SEQ ID NO: 6 | VFRREAHKSEIAHR |
| SEQ ID NO: 7 | EAHK |
| SEQ ID NO: 8 | DAHR |
| SEQ ID NO: 9 | DARK |
| SEQ ID NO: 10 | RDAHK |
| SEQ ID NO: 11 | RDAHKS |
| SEQ ID NO: 12 | RRDAHK |
| SEQ ID NO: 13 | RRDAHKS |
| SEQ ID NO: 14 | RDAHKSE |
| SEQ ID NO: 15 | RRDAHKSE |
| SEQ ID NO: 16 | FRRDAHKSEV |
| SEQ ID NO: 17 | FRRDAHKSEVA |
| SEQ ID NO: 18(HSA) | MKWVTFISLL FLFSSAYSRG VFRRDAHKSE |

VAHRFKDLGE ENFKALVLIA FAQYLQQCPF EDHVKLVNEV TEFAKTCVAD
ESAENCDKSL HTLFGDKLCT VATLRETYGE MADCCAKQEP ERNECFLQHK
DDNPNLPRLV RPEVDVMCTA FHDNEETFLK KYLYEIARRH PYFYAPELLF
FAKRYKAAFT ECCQAADKAA CLLPKLDELR DEGKASSAKQ RLKCASLQKF
GERAFKAWAV ARLSQRFPKA EFAEVSKLVT DLTKVHTECC HGDLLECADD
RADLAKYICE NQDSISSKLK ECCEKPLLEK SHCIAEVEND EMPADLPSLA
ADFVESKDVC KNYAEAKDVF LGMFLYEYAR RHPDYSVVLL LRLAKTYETT
LEKCCAAADP HECYAKVFDE FKPLVEEPQN LIKQNCELFE QLGEYKFQNA
LLVRYTKKVP QVSTPTLVEV SRNLGKVGSK CCKHPEAKRM PCAEDYLSVV
LNQLCVLHEK TPVSDRVTKC CTESLVNRRP CFSALEVDET YVPKEFNAET
FTFHADICTL SEKERQIKKQ TALVELVKHK PKATKEQLKA VMDDFAAFVE
KCCKADDKET CFAEEGKKLV AASQAALGL

| | |
|---|---|
| SEQ ID NO: 19 | DAKH |

What is claimed is:

1. A cell culture medium, comprising a peptide comprising superoxide dismutase activity and Cu+, Zn+ chelating activity.

2. The cell culture medium of claim 1, wherein the peptide is synthetically made.

3. The cell culture medium of claim 1 or claim 2, wherein the peptide comprises at least 4 amino acid residues.

4. The cell culture medium of any one of claims 1-3, wherein the peptide comprises at least one negatively-charged residue.

5. The cell culture medium of any one of claims 1-4, wherein the peptide comprises at least one positively-charged residue.

6. The cell culture medium of any one of claims 1-5, wherein the peptide is selected from the group consisting of DARK (SEQ ID NO:1), DTHK (SEQ ID NO: 2), and EAHK (SEQ ID NO:7).

7. The cell culture medium of any one of claims 1-6, wherein the peptide is present at a concentration between about 50 μg/mL and about 100 μg/ml

8. The cell culture medium of any of claims 1-7, wherein the medium is serum and albumin-free.

9. The cell culture medium of any of claims 1-8, further comprising a superoxide scavenger.

10. The cell culture medium of claim 9, wherein the superoxide scavenger comprises a compound containing (2,2,6,6-tertramethyl-1-yl)oxyl or variants thereof, or a flavonoid.

11. The cell culture medium of claim 10, wherein the superoxide scavenger comprises TEMPO, TEMPOL, or a variant thereof.

12. The cell culture medium of claim 9, wherein the superoxide scavenger comprises a compound that is not native to cells.

13. The cell culture medium of claim 9 or claim 10, wherein the superoxide scavenger comprises Mito-TEMPO.

14. The cell culture medium of any one of claims 1-13, further comprising a vitamin E or an analog thereof.

15. The cell culture medium of claim 14, wherein the vitamin E analog comprises a 6-chromanol group moiety.

16. The cell culture medium of claim 14, wherein the vitamin E or analog or variant thereof comprises α-tocopherol, β-tocopherol, γ-tocopherol, δ-tocopherol, α-tocotrienol, β-tocotrienol, δ-tocotrienol, γ-tocotrienol, α-tocoperol succinate, α-tocopherol monoglucoside, γ-tocopherol N,N-dimethyl glycine ester, or a substitution, isoform pure, racemic mixture, and/or mixture thereof.

17. The cell culture medium of claim 14, wherein the vitamin E analog is water soluble.

18. The cell culture medium of claim 15, wherein the vitamin E analog comprises 6-hydroxy-2,5,7,8-tetramethylchroman-2-carboxylic acid.

19. The cell culture medium of any one of claims 1-18, further comprising a hydrogen peroxide reducing reagent.

20. The cell culture medium of claim 19, wherein the hydrogen peroxide reducing agent comprises glutathione, N-acetyl cysteine, cysteine, sodium selenite, mannitol, a flavonoid, lipoic acid, or any combination thereof.

21. The cell culture medium of any one of claims 1-20, further comprising a stabilizer molecule.

22. The cell culture medium of claim 21, wherein the stabilizer molecule is trehalose or mannitol.

23. The cell culture medium of any one of claims 1-22, wherein the cell culture medium comprises at least one of a balanced salt solution, basal medium, and/or complex medium.

24. The cell culture medium of any one of claims 1-23, wherein the cell culture medium comprises at least one of: saline, phosphate-buffered saline, Dulbelcco's phosphate buffered saline, Hank's balanced salt solution, Earle's balanced salt solution, MEM, Opti-MEM, DMEM, CTS KnockOut DMEM, RPMI-1640, IMDM, Ham's F12, F-12 K, F-10, DMEM/F12, Neurobasal, McCoy's 5A medium, Leibowitz's L-15, Medium 199, Neurobasal A, Brainphys, GMEM, and/or William's E Medium.

25. A cell culture supplement, comprising a peptide comprising superoxide dismutase activity and Cu+, Zn+ chelating activity.

26. The cell culture supplement of claim 25, wherein the peptide is synthetically made.

27. The cell culture supplement of claim 25 or claim 26, wherein the peptide comprises at least 4 amino acid residues.

28. The cell culture supplement of any one of claims 25-27, wherein the peptide comprises at least one negatively-charged residue.

29. The cell culture supplement of any one of claims 21-28, wherein the peptide comprises at least one positively-charged residue.

30. The cell culture supplement of any one of claims 25-29, wherein the peptide is selected from the group consisting of DARK (SEQ ID NO:1) and DTHK (SEQ ID NO:2).

31. The cell culture supplement of any of claims 25-30, further comprising a superoxide scavenger.

32. The cell culture supplement of claim 31, wherein the superoxide scavenger comprises a compound containing (2,2,6,6-tertramethyl-1-yl)oxyl or variants thereof, or a flavonoid

33. The cell culture supplement of claim 32, wherein the superoxide scavenger comprises TEMPO, TEMPOL, or a variant thereof.

34. The cell culture supplement of claim 31, wherein the superoxide scavenger comprises a compound that is not native to cells.

35. The cell culture supplement of claim 31 or claim 32, wherein the superoxide scavenger comprises Mito-TEMPO.

36. The cell culture supplement of any one of claims 25-35, further comprising a vitamin E or an analog thereof.

37. The cell culture supplement of claim 36, wherein the vitamin E analog comprises a 6-chromanol group moiety.

38. The cell culture supplement of claim 36, wherein the vitamin E or analog or variant thereof comprises α-tocopherol, β-tocopherol, γ-tocopherol, δ-tocopherol, α-tocotrienol, β-tocotrienol, δ-tocotrienol, γ-tocotrienol, α-tocoperol succinate, α-tocopherol monoglucoside, γ-tocopherol N,N-dimethyl glycine ester, or a substitution, isoform pure, racemic mixture, and/or mixture thereof.

39. The cell culture supplement of any one of claims 36-38, wherein the vitamin E analog is water soluble.

40. The cell culture supplement of claim 37, wherein the vitamin E analog comprises 6-hydroxy-2,5,7,8-tetramethylchroman-2-carboxylic acid.

41. The cell culture supplement of any one of claims 25-40, further comprising a hydrogen peroxide reducing reagent.

42. The cell culture supplement of claim 41, wherein the hydrogen peroxide reducing agent comprises glutathione, N-acetyl cysteine, cysteine, sodium selenite, mannitol, a flavonoid, lipoic acid, or any combination thereof.

43. The cell culture supplement of any one of claims 25-42, further comprising a stabilizer molecule.

44. The cell culture supplement of claim 43, wherein the stabilizer molecule is trehalose or mannitol.

45. The cell culture supplement of any one of claims 25-44, wherein the cell culture supplement is serum-free.

46. The cell culture supplement of any one of claims 25-45, wherein the cell culture supplement is albumin-free.

47. The cell culture supplement of any one of claims 25-46, wherein the supplement is added to a serum free cell culture medium.

48. A cell culture supplement, comprising:
(i) a peptide comprising superoxide dismutase activity and Cu+, Zn+ chelating activity;
(ii) vitamin E or an analog thereof; and
(iii) a superoxide scavenger.

49. A method for growing cells in culture, comprising growing the cells in a cell culture medium according to any one of claims 1-24.

50. A method of growing cells in culture, comprising growing the cells in a cell culture medium supplemented with a cell culture supplement according to any one of claims 25-48.

51. A method of rescuing cells from albumin-induced toxicity, comprising contacting a cell exhibiting albumin-induced toxicity with a cell culture supplement according to any one of claims 25-48.

52. A method for expanding cells in culture, comprising contacting the cells with a serum-free, albumin-free cell culture medium in a cell culture medium according to any one of claims 1-24.

53. A method for recovering cells from oxidative stress, comprising contacting the cells with a cell culture supplement according to any one of claims 25-48 or growing the cells in a cell culture medium according to any one of claims 1-24.

54. The method of claim 53, wherein the oxidative stress was from freezing the cells.

55. The method of claim 53, wherein the oxidative stress was exposure to a lipid rich condition.

56. The method of any one of claims 49-55, wherein the cells are therapeutic cells.

57. The method of any one of claims 49-56, wherein the cells are selected from the group consisting of: mesenchymal stem cells, neural progenitor cells, retinal pigment epithelium, pancreatic beta cells, cardiac muscle cells, HEK-293 cells, and CHO cells.

58. A cell culture kit comprising a serum-free cell culture medium and the cell culture supplement of any one of claims 25-46.

59. The cell culture kit of claim 58, wherein the cell culture supplement comprises:
(i) a peptide comprising superoxide dismutase activity and Cu+, Zn+ chelating activity;
(ii) vitamin E or an analog thereof; and
(iii) a superoxide scavenger, and
wherein (i)-(iii) are provided in separate containers.

60. The kit of claim 58, wherein the wherein the cell culture supplement comprises:
(i) a peptide comprising superoxide dismutase activity and Cu+, Zn+ chelating activity;
(ii) vitamin E or an analog thereof; and
(iii) a superoxide scavenger, and
wherein (i)-(iii) are provided in a single container.

61. The kit of claim 59 or claim 60, further comprising a hydrogen peroxide reducing reagent.

62. The kit of claim 61, wherein the hydrogen peroxide reducing reagent comprises glutathione and/or lipoic acid, or variant thereof.
